(19) United States
(12) Patent Application Publication
(10) Pub. No.: US 2026/0263435 A1
(43) Pub. Date: Sep. 10, 2026

(54) AXITINIB COMPOSITIONS AND METHODS OF TREATING OCULAR DISORDERS

(71) Applicant: Clearside Biomedical, Inc., Alpharetta, GA (US)

(72) Inventors: Viral KANSARA, Alpharetta, GA (US); Thomas CIULLA, Alpharetta, GA (US); Colette HALL, Alpharetta, GA (US); Barry KAPIK, Alpharetta, GA (US)

(21) Appl. No.: 19/351,042

(22) Filed: Oct. 6, 2025

Related U.S. Application Data
(60) Provisional application No. 63/704,375, filed on Oct. 7, 2024.

(51) Int. Cl. A61K 31/4439; A61P 27/02

(52) U.S. Cl. CPC A61K 31/4439; A61P 27/02

(57) ABSTRACT

This disclosure is generally related to ophthalmic therapies, and more particularly to methods and devices that allow for infusion of a fluid drug formulation into posterior ocular tissues for targeted, localized treatment, for example, for the treatment of wet AMD in human patients. The drug formulation may be injected into the suprachoroidal space (SCS) or subretinal space of an eye. The drug formulation may include a tyrosine kinase inhibitor, such as axitinib.

### CROSS-REFERENCE TO RELATED APPLICATIONS

This application claims the benefit of priority to U.S. provisional application No. 63/704,375, filed on Oct. 7, 2024, which is hereby incorporated by reference in its entirety.

### BACKGROUND OF THE INVENTION

Neovascular age-related macular degeneration (nAMD), also known as “wet” AMD, is the leading cause of vision loss in the industrialized world. It is caused by abnormal blood vessel growth in the choriocapillaris, a layer of capillaries situated immediately below Bruch's membrane. Choroidal neovascularization leads to the leakage of blood, lipids, and serum into the retinal layers, which can result in permanent damage to light-sensitive retinal cells and irreversible central vision distortions.

Although not fully elucidated, much is known regarding the pathogenesis of wet AMD. The vascular endothelial growth factor (VEGF) signaling pathway has been shown to be centrally involved in the 10% to 15% of AMD diagnoses classified as the neovascular type (wet AMD). In this pathway, VEGF signaling ligands bind to different isoforms of VEGF receptors (VEGFR) to activate cellular processes that promote growth of new vasculature. Specifically, VEGF-A, which acts at VEGFR-1 and -2, has been shown to promote abnormal blood vessel growth and is therefore an optimal target for treatment. Currently, anti-VEGF-A drugs are the standard of care for the treatment of wet AMD; however, an unmet need remains for significantly improving and maintaining visual acuity in patients.

Choroidal neovascularization (CNV) is the creation of new blood vessels in the choroid layer of the eye. Choroidal neovascularization is a common cause of, and/or can be associated with, wet AMD. CNV can occur rapidly in individuals with defects in Bruch's membrane, the innermost layer of the choroid. It is also associated with excessive amounts of VEGF. CNV can also occur frequently with the rare genetic disease pseudoxanthoma elasticum and rarely with the more common optic disc drusen. CNV has also been associated with extreme myopia or malignant myopic degeneration, where in choroidal neovascularization occurs primarily in the presence of cracks within the retinal macular tissue known as lacquer cracks. CNV can create a sudden deterioration of central vision, noticeable within a few weeks. Other symptoms which can occur include color disturbances, and metamorphopsia (distortions in which straight lines appears wavy). Hemorrhaging of the new blood vessels can accelerate the onset of symptoms of CNV.

Currently, even the most successful treatments of wet AMD and CNV do not preclude reoccurrence, making multiple treatments likely. Current treatments require life-long, frequent injections to maintain efficacy, and such treatment regimen tends to cause a treatment burden for patients resulting in reduced compliance and under-treatment leading to potentially limited outcomes. In addition, currently available treatments do not restore vision that has already been lost. Therefore, there is a need in the art for treatment breakthroughs, in order to maintain vision for a longer period of time without repeated laser or therapeutic treatments.

### SUMMARY OF THE INVENTION

Methods and compositions for the treatment of neovascular age-related macular degeneration (nAMD; also referred to herein as wet AMD), choroidal neovascularization (CNV), wet AMD associated with CNV, and related diseases are provided. The compositions comprise one or more tyrosine kinase inhibitors and are delivered to the suprachoroidal space of the eye via a non-surgical means. In some embodiments the tyrosine kinase inhibitor has activity against vascular endothelial growth factor (VEGF) and/or platelet derived growth factor (PDGF). In some embodiments, the tyrosine kinase inhibitor is axitinib. In some embodiments, the present disclosure provides an axitinib formulation, CLS-AX.

In some embodiments, the present disclosure provides methods for treating wet AMD, comprising non-surgically administering a formulation comprising an effective amount of axitinib to the suprachoroidal space of an eye of a patient in need thereof. In some embodiments, the method comprises administering about 1.0 mg of axitinib to the eye.

In some embodiments, the formulation comprising the effective amount of axitinib is administered in a first dose. In some embodiments, the method further comprises administering one or more maintenance doses. In some embodiments, at least one of the one or more maintenance doses is administered about 2 months to about 8 months following the first dose. In some embodiments, at least one of the one or more maintenance doses is administered about 3 months to about 6 months following the first dose. In some embodiments, at least one of the one or more maintenance doses is administered at about 3 months, at about 4 months, at about 5 months, or at about 6 months, following the first dose. In some embodiments, at least one of the one or more maintenance doses is administered about every 4-36 weeks, about every 6-32 weeks, about 8-28 weeks, about 12-24 week, or about every 24 weeks following the first dose.

In some embodiments, the method reduces injection frequency for the human patient within about 24 weeks to about 36 weeks following the first dose compared to a human patient who is not administered the formulation comprising the effective amount of axitinib to the SCS. In some embodiments, the method reduces injection frequency for the human patient within about 24 weeks to about 36 weeks following the first dose compared to the injection frequency of the human patient prior to administration of the formulation. In some embodiments, the injection frequency is reduced to about 30-90% fewer doses. In some embodiments, the injection frequency is reduced to about 1-4 fewer injections. In some embodiments, the human patient does not require a maintenance dose within about 3 months, 4 months, or 6 months following the first dose, determined by Disease Activity Assessment (DAA). In some embodiments, the human patient requires only one maintenance dose within about 3 months, 4 months, or 6 months following the first dose, determined by Disease Activity Assessment (DAA). In some embodiments, the human patient does not require an additional VEGF therapy following about 3 months, 4 months, or 6 months following the last administration of the formulation, determined by Disease Activity Assessment (DAA). In some embodiments, the additional VEGF therapy is intravitreal aflibercept or intravitreal faricimab treatment.

In some embodiments, the human patient was diagnosed with wet AMD determined by BCVA measurement of 20 to 80 letters prior to administration of the formulation. In some embodiments, the human patient has been previously treated for wet AMD or wet AMD associated choroidal neovascularization (CNV). In some embodiments, the human patient was previously treated with a VEGF antagonist. In some embodiments, the VEGF antagonist is aflibercept, faricimab, bevacizumab, ranibizumab, or pegaptinib sodium. In some embodiments, the aflibercept is administered at a dose of about 2 mg. In some embodiments, the faricimab is administered at a dose of about 6 mg. In some embodiments, the VEGF antagonist is administered via intravitreal injection. In some embodiments, the VEGF antagonist has been administered for about 2 to 4 times within about 4 to about 6 months prior to administration of the formulation. In some embodiments, the method reduces the injection frequency within about 24 weeks to about 36 weeks following the first dose compared to a method of administering 2 mg of aflibercept. In some embodiments, the injection frequency is reduced to about 30-90% fewer doses compared to the method of administering 2 mg of aflibercept. In some embodiments, the injection frequency is reduced to 1-4 fewer injections compared to the method of administering 2 mg of aflibercept.

In some embodiments, the patient loses no more than 10 letters as measured by best-corrected visual acuity (BCVA) measurement, compared to the patient's BCVA measurement prior to administration of the formulation. In some embodiments, the patient loses no more than 5 letters as measured by best-corrected visual acuity (BCVA) measurement, compared to the patient's BCVA measurement prior to administration of the formulation. In some embodiments, the patient experiences no loss in vision as measured by BCVA, compared to the patient's BCVA measurement prior to administration of the formulation. In some embodiments, the patient experiences an improvement in vision, as measured by gaining ≥5 letters, ≥10 letters or ≥15 letters in BCVA measurement, compared to the patient's BCVA prior to administration of the formulation. In some embodiments, the patient experiences the improvement in BCVA for at least 2, 3, 4, 5, 6, 7, 8, or 9 months after administration of the formulation comprising axitinib. In some embodiments, the patient experiences minimal loss in vision for 2, 3, 4, 5, 6, 7, 8, or 9 months after administration of the formulation comprising axitinib, wherein the minimal loss of vision is losing no more than 2 letters as measured by BCVA. In some embodiments, the patient experiences minimal loss in vision for 2, 3, 4, 5, 6, 7, 8, or 9 months after administration of the formulation comprising axitinib, wherein the minimal loss of vision is losing no more than 5 letters as measured by BCVA. In some embodiments, the patient does not experience a loss of vision within 2, 3, 4, 5, 6, 7, 8, or 9 months after the administration of the formulation comprising axitinib, wherein the vision is measured by BCVA.

In some embodiments, the present disclosure provides methods that result in a decrease in retinal thickness in the treated eye, as measured by Spectral Domain Optical Coherence Tomography (SD-OCT) compared to the patient's retinal thickness in the eye in need of treatment prior to the administration of the formulation. In some embodiments, the decrease in retinal thickness is ≥25 μm, ≥50 μm, ≥75 μm or ≥100 μm. In some embodiments, the decrease in retinal thickness is ≥5%, ≥10% or ≥25%. In some embodiments, the method results in a decrease in retinal thickness in the eye, as measured by SD-OCT, compared to the retinal thickness in the eye of a patient who was not administered a formulation comprising axitinib. In some embodiments, the patient does not exhibit an increase in retinal thickness in the eye within 24 weeks or 36 weeks after the administration of the formulation comprising axitinib. In some embodiments, the patient does not exhibit an increase in retinal thickness in the eye within 2, 3, 4, 5, 6, 7, 8, or 9 months after the administration of the formulation comprising axitinib.

In some embodiments, the axitinib formulation comprises about 1 mg/ml axitinib. In some embodiments, the axitinib formulation comprise about 0.01% to about 0.05% polysorbate 80, about 0.5% sodium carboxymethylcellulose, about 0.79% sodium chloride, about 0.06% sodium phosphate monobasic, and about 0.08% sodium phosphate dibasic.

### BRIEF DESCRIPTION OF THE DRAWINGS

FIG. 1 shows the 6-month extension study design including dosing schedules for prior anti-VEGF therapies and any additional therapies after Day 1 suprachoroidal injection with CLS-AX as described in Example 3.

FIG. 2 shows the 6-month extension study design including dosing schedules for prior anti-VEGF therapies and additional therapies after Day 1 suprachoroidal injection with CLS-AX, per protocol criteria as described in Example 3.

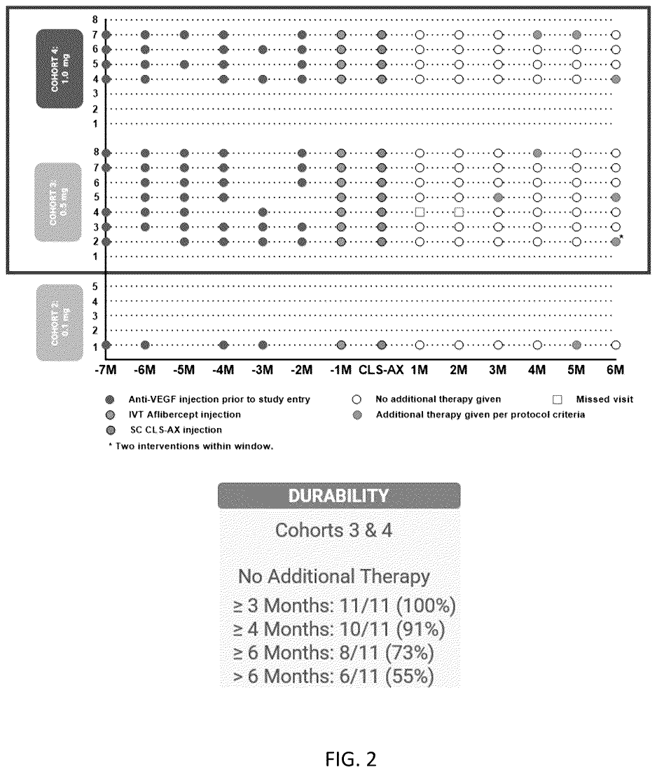

FIG. 3 shows the mean (±SEM) best corrected visual acuity letter score (ETDRS letters) changes from screening in cohorts 3 and 4 over a 6-month study period as described in Example 3.

FIG. 4 shows the mean (±SEM) best corrected visual acuity letter score (ETDRS letters) changes from screening in cohorts 3 and 4 over a 6-month study period excluding data after additional treatment as described in Example 3.

FIG. 5 shows the mean (±SEM) central subfield thickness changes from screening in cohorts 3 and 4 over a 6-month study period as described in Example 3.

FIG. 6 shows the mean (±SEM) central subfield thickness changes from screening in cohorts 3 and 4 over a 6-month study period excluding data after additional treatment as described in Example 3.

FIG. 7 shows the study design and dosing schedules of the clinical study as described in Example 4.

FIG. 8 shows the mean (±SEM) best corrected visual acuity letter score (BCVA letters) changes from baseline to Week 24 and Week 36 in CLS-AX and aflibercept arms as described in Example 4.

FIG. 9 shows the mean (±SEM) central subfield retinal thickness (CSRT) changes from baseline to Week 24 and Week 36 in CLS-AX and aflibercept arms as described in Example 4.

FIG. 10 shows the mean (±SEM) BCVA letter and CSRT changes from baseline to Week 24 and Week 36 in sub-groups of CLS-AX arm participants who did not require supplemental therapy as described in Example 4.

FIG. 11 shows the % of participants who did not require additional treatment at Week 12, Week 16, Week 20, and Week 24 visits as described in Example 4.

FIG. 12 shows the % of participants who did not require additional treatment at Week 12, Week 16, Week 20, and Week 24 visits according to the disease activity assessment (DAA) criteria, as described in Example 4.

### DETAILED DESCRIPTION OF THE INVENTION

This disclosure is generally related to ophthalmic therapies, and more particularly to compositions and methods that allow for infusion of a fluid drug formulation into posterior ocular tissues for targeted, localized treatment, for example, for the treatment of diseases and disorders of the eye associated with neovascularization. In some embodiments, the diseases and disorders include neovascular age-related macular degeneration (nAMD; also referred to herein as wet AMD), nonexudative AMD, choroidal neovascularization (CNV), retinal vein occlusion (RVO), wet AMD associated with RVO, and/or wet AMD associated with CNV.

In some embodiments, the formulations comprise one or more tyrosine kinase inhibitors and are administered to the suprachoroidal space (SCS) of the eye via a non-surgical means, for example via a hollow microneedle, and/or an injection device comprising a hollow needle wherein the needle has an effective length of about 500 to about 2000 microns. The methods and formulations provided herein allow for effective posterior segment drug delivery, and generally embody the following characteristics: (1) the methods are non-surgical and thus minimally invasive and safe; (2) the drug formulations are administered in such a way that they are well targeted to the posterior segment of the eye and/or the suprachoroidal space (SCS) of the eye and/or the supraciliary space of the eye and/or the supraretinal space of the eye and/or the subretinal space of the eye, while simultaneously limiting drug exposure to the anterior segment or other regions of the eye; (3) the methods and formulations are capable of delivering drug in a sustained and/or controlled manner; (4) the methods and devices are user-friendly. The non-surgical SCS delivery methods and drug formulations for SCS delivery set forth herein achieve these desired characteristics.

Axitinib is a tyrosine kinase inhibitor (TKI) that antagonizes the vascular endothelial growth factor receptors VEGFR-1, VEGFR-2, and VEGFR-3, as well as of the platelet-derived growth factor receptors (PDGFR) and c-Kit receptors. Axitinib was initially approved in 2012 as an oral tablet formulation (INLYTA®) at a dose of 5 mg given twice daily for the treatment of advanced renal cell carcinoma after failure of one prior systemic therapy. An axitinib formulation suitable for delivery to the eye is provided herein. In some embodiments, the axitinib formulation suitable for delivery to the eye provided herein is “CLS-AX.” Exemplary CLS-AX formulations are provided in Table 1A and Table 1B.

The term “suprachoroidal space,” is used interchangeably herein with suprachoroidal, SCS, suprachoroid, suprachoroidia, and the like; and describes the potential space in the region of the eye disposed between the sclera and choroid. This region primarily is composed of closely packed layers of long pigmented processes derived from each of the two adjacent tissues; however, a space can develop in this region as a result of fluid or other material buildup in the suprachoroidal space and the adjacent tissues. Those skilled in the art will appreciate that the suprachoroidal space frequently is expanded by fluid buildup because of some disease state in the eye or as a result of some trauma or surgical intervention. In the present description, however, in some embodiments, the fluid buildup is intentionally created by infusion of a drug formulation into the suprachoroid to create the suprachoroidal space (which is filled with drug formulation). Not wishing to be bound by theory, it is believed that the SCS region serves as a pathway for uveoscleral outflow (i.e., a natural process of the eye moving fluid from one region of the eye to the other through) and becomes a real space in instances of choroidal detachment from the sclera.

As used herein, “non-surgical” ocular drug delivery devices and methods refer to methods and devices for drug delivery that do not require general anesthesia and/or retrobulbar anesthesia (also referred to as a retrobulbar block). Alternatively or additionally, a “non-surgical” ocular drug delivery method is performed with an instrument having a needle with an effective length of less than 2000 microns; and or an instrument having a needle with a diameter of 28 gauge or smaller. In some embodiments, the present disclosure provides a method of administering the pharmaceutical composition with a needle with a diameter of 28 gauge or 30 gauge. In some embodiments, the needle has a diameter of 30 G. Alternatively or additionally, “non-surgical” ocular drug delivery methods do not require a guidance mechanism that is typically required for ocular drug delivery via a shunt or cannula. Non-surgical ocular drug delivery methods provided herein may be used in a clinic or out-patient setting and do not require a hospital setting. As used herein, “surgical” ocular drug delivery includes insertion of devices or administration of drugs by surgical means, for example, via incision to expose and provide access to regions of the eye including the posterior region, and/or via insertion of a stent, shunt, or cannula and/or via a method that requires anesthesia (e.g., general or retrobulbar anesthesia).

The surgical and non-surgical posterior ocular disorder treatment methods and devices described herein are particularly useful for the local delivery of drugs to the posterior region of the eye, for example the retinochoroidal tissue, macula, retinal pigment epithelium (RPE) and optic nerve in the posterior segment of the eye. In one embodiment, the non-surgical methods provided herein can be used to target drug delivery to specific posterior ocular tissues or regions within the eye or in neighboring tissue. In one embodiment, the methods described herein deliver drug specifically to the sclera, the choroid, the Brach's membrane, the retinal pigment epithelium, the subretinal space, the retina, the macula, the optic disk, the optic nerve, the ciliary body, the trabecular meshwork, the aqueous humor, the vitreous humor, and/or other ocular tissue or neighboring tissue in the eye of a human patient in need of treatment. The methods provided herein, in one embodiment, can be used to target drug delivery to specific posterior ocular tissues or regions within the eye or in neighboring tissue.

As provided throughout, in one embodiment, the methods described herein are carried out with a puncture member, which may comprise a hollow or solid microneedle, for example, a rigid microneedle. As used herein, the term “microneedle” refers to a conduit body having a base, a shaft, and a tip end suitable for insertion into the sclera and other ocular tissue and has dimensions suitable for minimally invasive insertion and drug formulation infusion as described herein. Both the “length” and “effective length” of the microneedle encompass the length of the shaft of the microneedle and the bevel height of the microneedle. In some embodiments, the needles used herein are microneedles in that they have an effective length of less than 2000 microns. For example, in some embodiments, the needles useful in the methods described herein are microneedles in that they have an effective length of about 50 microns to about 2000 microns, or about 500 microns to about 1800 microns, or about 700 microns to about 1500 microns, or about 900 microns to about 1200 microns. In some embodiments, the needles useful in the methods described herein are microneedles in that they have an effective length of about 800 microns, about 900 microns, about 1000 microns, about 1100 microns, or about 1200 microns. In some embodiments, the device used to carry out the methods described herein comprises one of the devices disclosed in U.S. Pat. No. 9,539,139, issued Jan. 10, 2017 or International Patent Application Publication No. WO2014/179698 (Application No. PCT/US2014/036590), filed May 2, 2014 and entitled “Apparatus and Method for Ocular Injection,” each of which is incorporated by reference herein in its entirety for all purposes. In some embodiments, the device used to carry out the methods described herein comprises one of the devices disclosed in International Patent Application Publication No. WO2014/036009 (Application No. PCT/US2013/056863), filed Aug. 27, 2013 and entitled “Apparatus and Method for Drug Delivery Using Microneedles,” incorporated by reference herein in its entirety for all purposes. In some embodiments, the microneedle is an SCS microinjector as described herein.

As used herein, the term “hollow” includes a single, straight bore through the center of the microneedle, as well as multiple bores, bores that follow complex paths through the microneedles, multiple entry and exit points from the bore(s), and intersecting or networks of bores. That is, a hollow microneedle has a structure that includes one or more continuous pathways from the base of the microneedle to an exit point (opening) in the shaft and/or tip portion of the microneedle distal to the base. In some embodiments, the microneedle further comprises additional catheter or cannula inside of the hollow needle.

The microneedle device in one embodiment, comprises a fluid reservoir for containing the therapeutic formulation (e.g., drug or cell formulation), e.g., as a solution or suspension, and the drug reservoir (which can include any therapeutic formulation) being in operable communication with the bore of the microneedle at a location distal to the tip end of the microneedle. The fluid reservoir may be integral with the microneedle, integral with the elongated body, or separate from both the microneedle and elongated body.

In some embodiments, features of the devices, formulations, and methods are provided in U.S. Pat. No. 9,636,332, U.S. Patent Application Publication No. 2018-0042765, International Patent Application Publication Nos. WO2014/074823 (Application No. PCT/US2013/069156), WO2015/195842 (Application No. PCT/US2015/036299), U.S. Patent Publication No. 2019-0269702, WO 2017/120600 (Application No. PCT/US2017/012755), and/or WO2017/120601 (Application No. PCT/US2017/012757), each of which is hereby incorporated by reference in its entirety for all purposes.

In one embodiment, the device used to carry out one of the methods described herein comprises the device described in U.S. Design patent application Ser. No. 29/506,275 entitled, “Medical Injector for Ocular Injection,” filed Oct. 14, 2014, the disclosure of which is incorporated herein by reference in its entirety for all purposes. In one embodiment, the device used to carry out one of the methods described herein comprises the device described in U.S. Patent Publication No. 2015/0051581 or U.S. Patent Publication No. 2017/0095339, which are each incorporated herein by reference in their entireties for all purposes. In some embodiments, such a device is an SCS microinjector as described herein.

As used herein, the terms “about” and “approximately” generally mean plus or minus 10% of the value stated. For example, about 0.5 would include 0.45 and 0.55, about 10 would include 9 to 11, about 1000 would include 900 to 1100.

Further details of possible manufacturing techniques for the microneedles and/or microinjectors provided herein are described, for example, in U.S. Patent Application Publication No. 2006/0086689, U.S. Patent Application Publication No. 2006/0084942, U.S. Patent Application Publication No. 2005/0209565, U.S. Patent Application Publication No. 2002/0082543, U.S. Pat. Nos. 6,334,856, 6,611,707, 6,743,211 and PCT/US2014/36590, filed May 2, 2014, all of which are incorporated herein by reference in their entireties for all purposes.

Any of the methods described herein can be performed use any suitable injector of the types shown and described herein. In some embodiments, in accordance with the methods described herein, the dose of drug has a delivered volume of at least about 20 μL, at least about 50 μL, at least about 100 μL, at least about 200 μL or at least about 500 μL. In one embodiment, the amount of therapeutic formulation delivered into the suprachoroidal space from the devices described herein is from about 10 μL to about 200 μL, e.g., from about 50 μL to about 150 μL. In another embodiment, from about 10 μL to about 500 μL, e.g., from about 50 μL to about 250 μL, is non-surgically administered to the suprachoroidal space. In some embodiments, about 100 μL of a drug formulation is non-surgically administered to the suprachoroidal space. In some embodiments, the drug formulation comprises 100 μL of an axitinib formulation, e.g., CLS-AX.

The SCS drug delivery methods provided herein allow for the delivery of drug formulation over a larger tissue area and to more difficult to target tissue in a single administration as compared to previously known needle devices. Not wishing to be bound by theory, it is believed that upon entering the SCS the drug formulation flows circumferentially from the insertion site toward the retinochoroidal tissue, macula, and optic nerve in the posterior segment of the eye as well as anteriorly toward the uvea and ciliary body. In addition, a portion of the infused drug formulation may remain in the SCS as a depot, or remain in tissue overlying the SCS, for example the sclera, near the microneedle insertion site, serving as additional depot of the drug formulation that subsequently can diffuse into the SCS and into other adjacent posterior tissues.

The term “subject” is used interchangeably herein with the term “patient.” The subject may be any mammal. Preferably, the subject is a human patient. The human patient treated with the methods and devices provided herein may be an adult or a child. In one embodiment, the subject presents with a retinal thickness of greater than 300 μm (e.g., central subfield thickness as measured by optical coherence tomography). In another embodiment, the patient in need of treatment has a BCVA score of ≥20 letters read in each eye (e.g., 20/400 Snellen approximate). In yet another embodiment, the patient in need of treatment has a BCVA score of ≥20 letters read in each eye (e.g., 20/400 Snellen approximate), but ≤70 letters read in the eye in need of treatment.

In some embodiments, the patient is a human patient who is diagnosed with wet AMD. In some embodiments, the human patient is diagnosed with wet AMD determined by BCVA score measurement of 20 to 80 letters prior to treatment. In some embodiments, the human patient is considered as a “difficult-to-treat” patient who is diagnosed with active wet AMD, choroidal neovascularization (CNV), or wet AMD associated with CNV. In some embodiments, the human patient has been previously treated for wet AMD or CNV.

The term “comparable patient” or “comparable subject” are used interchangeably herein and refers to a patient whose physical characteristics relevant to dosing are expected to be substantially the same as that of the patient who received the axitinib treatment.

Therapeutic response, in one embodiment, is assessed via a visual acuity measurement at one and/or two months post treatment (e.g., by measuring the mean change in best corrected visual acuity (BCVA) from baseline, i.e., prior to treatment). In one embodiment, a patient treated by one or more of the methods provided herein experiences an improvement in BCVA from baseline, at any given time point (e.g., 2 weeks after administration, 4 weeks after administration, 2 months after administration, 3 months after administration), of at least 2 letters, at least 3 letters, at least 4 letters, at least 5 letters, at least 6 letters, at least 7 letters, at least 8 letters, at least 9 letters, at least 10 letters, at least 11 letters, at least 12 letters, at least 13 letters, at least 14 letters, at least 15 letters, at least 16 letters, at least 17 letters, at least 18 letters, at least 19 letters, at least 20 letters, and all values in between, as compared to the patient's BCVA prior to administration of axitinib. In some embodiments, the patient experiences an improvement in vision, as measured by gaining ≥5 letters, ≥10 letters or ≥15 letters in BCVA measurement, compared to the patient's BCVA prior to administration of the formulation.

In one embodiment, the patient gains about 5 letters or more, 6 letters or more, 7 letters or more, 8 letters or more, 9 letters or more, about 10 letters or more, 11 letters or more, 12 letters or more, 13 letters or more, 14 letters or more, 15 letters or more, 16 letters or more, 17 letters or more, 18 letters or more, 19 letters or more, about 20 letters or more, 21 letters or more, 22 letters or more, 23 letters or more, 24 letters or more, or about 25 letters or more in a BCVA measurement after administration of axitinib, compared to the patient's BCVA measurement prior to undergoing treatment. In even a further embodiment, the patient gains from about 5 to about 30 letters, 10 to about 30 letters, from about 15 letters to about 25 letters or from about 15 letters to about 20 letters in a BCVA measurement, compared to the patient's BCVA measurement prior to treatment with axitinib. In one embodiment, the BCVA gain is about 2 weeks, about 1 month, about 2 months, about 3 months, about 4 months, about 5 months, or about 6 months after administration of axitinib. In another embodiment, the BCVA is measured at least about 2 weeks, at least about 1 month, at least about 2 months, at least about 3 months, at least about 4 months, about 5 months, or at least about 6 months after administration of axitinib.

In one embodiment, the BCVA is based on the Early Treatment of Diabetic Retinopathy Study (ETDRS) visual acuity charts and is assessed at a starting distance of 4 meters.

In another embodiment, the patient subjected to a treatment method, e.g., with one of the devices provided herein substantially maintains his or her vision subsequent to the treatment (e.g., a single administration or multiple administrations of axitinib to the suprachoroidal space of the eye), as measured by losing fewer than 15 letters in a best-corrected visual acuity (BCVA) measurement, compared to the patient's BCVA measurement prior to undergoing treatment. In a further embodiment, the patient loses fewer than 10 letters, fewer than 9 letters, fewer than 8 letters, fewer than 7 letters, fewer than 6 letters, fewer than 5 letters, fewer than 4 letters, fewer than 3 letters, or fewer than 2 letters in a BCVA measurement, compared to the patient's BCVA measurement prior to undergoing treatment. In some embodiments, the patient experiences no further loss of vision subsequent to treatment with axitinib (e.g., the patient experiences a loss of 0 letter as measured by BCVA). In some embodiments, the patient experiences a gain in vision subsequent to treatment (e.g., a single administration or multiple administrations of axitinib to the suprachoroidal space of the eye). For example, in some embodiments, the patient experiences a gain of at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or more letters. In some embodiments, “minimal loss of vision” as used herein means losing no more than 1 letter, no more than 2 letters, no more than 3 letters, no more than 4 letters, no more than 5 letters, no more than 6 letters, no more than 7 letters, no more than 8 letters, no more than 9, letters, no more than 10 letters, no more than 11 letters, no more than 12 letters, no more than 13 letters, no more than 14 letters, or no more than 15 letters. In some embodiments, the patient experiences a minimal loss of vision, relative to the patient's baseline vision prior to treatment, over 2, 6, 12, 18, or 24 months. In some embodiments, the patient experiences minimal loss in vision for 2, 3, 4, 5, 6, 7, 8, or 9 months after administration of the formulation comprising axitinib, wherein the minimal loss of vision is losing no more than 1, 2, 3, 4, 5, 6, 7, 8, 9, 10 letters as measured by BCVA. In some embodiments, the patient experiences minimal loss in vision for 2, 3, 4, 5, 6, 7, 8, or 9 months after administration of the formulation comprising axitinib, wherein the minimal loss of vision is losing no more than 2 letters as measured by BCVA. In some embodiments, the patient experiences minimal loss in vision for 2, 3, 4, 5, 6, 7, 8, or 9 months after administration of the formulation comprising axitinib, wherein the minimal loss of vision is losing no more than 5 letters as measured by BCVA. In some embodiments, the patient does not experience a loss of vision within 2, 3, 4, 5, 6, 7, 8, or 9 months after the administration of the formulation comprising axitinib, wherein the vision is measured by BCVA.

In some embodiments, the patient experiences the improvement in BCVA for at least 2, 3, 4, 5, 6, 7, 8, or 9 months after administration of the formulation comprising axitinib. In some embodiments, the patient experiences a gain in vision, relative to the patient's baseline vision prior to treatment, over 2, 6, 12, 18, or 24 months.

A decrease in retina thickness and/or macula thickness is one measurement of treatment efficacy of the methods provided herein. For example, in one embodiment, patient suffering from wet AMD treated by one of the methods provided herein (e.g., administration of a drug (e.g., axitinib) to the suprachoroidal space of an eye) experiences a decrease in retinal thickness from baseline. For example, in one embodiment, the patient experiences a decrease in central subfield thickness (CST) at any given time point after administration of the drug, e.g., a decrease of least about 20 μm, or at least about 40 μm, or at least about 50 μm, or at least about 100 μm, or at least about 150 μm or at least about 200 μm, or from about 50-100 μm, and all values in between. In another embodiment, the patient experiences a ≥5%, ≥10%, ≥15%, ≥20%, ≥25% decrease in retinal thickness (e.g., CST) subsequent to administration of the drug. In some embodiments, the patient experiences a decrease in CST, relative to the patient's baseline CST prior to treatment, for at least 2, 6, 12, 18, or 24 months.

In one embodiment, a patient treated by the methods provided herein experiences a decrease in retinal thickness from baseline (i.e., retinal thickness prior to treatment), at any given time point, of from about 20 μm to about 200 μm, at from about 40 μm to about 200 μm, of from about 50 μm to about 200 μm, of from about 100 μm to about 200 μm, or from about 150 μm to about 200 μm. In one embodiment, change in retinal thickness from baseline is measured as a change in CST, for example, by spectral domain optical coherence tomography (SD-OCT). In some embodiments, the decrease in retinal thickness is ≥25 μm, ≥50 μm, ≥75 μm or ≥100 μm. In some embodiments, the decrease in retinal thickness is ≥5%, ≥10% or ≥25%. In some embodiments, the method results in a decrease in retinal thickness in the eye, as measured by SD-OCT, compared to the retinal thickness in the eye of a patient who was not administered a formulation comprising axitinib. In some embodiments, the patient does not exhibit an increase in retinal thickness in the eye within 2, 3, 4, 5, 6, 7, 8, or 9 months after the administration of the formulation comprising axitinib. In some embodiments, the patient does not exhibit an increase in retinal thickness in the eye within 12 weeks after the administration of the formulation comprising axitinib.

In one embodiment, the decrease in retinal thickness is measured about 2 weeks, about 1 month, about 2 months, about 3 months, about 6 months, about 12 months, about 18 months, or about 24 months after administration of the drug. In another embodiment, the decrease in retinal thickness is measured at least about 2 weeks, at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 18 months, or at least about 24 months after administration of the drug. In one embodiment, where multiple dosing sessions are employed, a decrease in retinal thickness is sustained by the patient for at least about 2 weeks, at least about 1 month, at least about 2 months, at least about 3 months, at least about 4 months, at least about 5 months, or at least about 6 months after each drug administration.

A reduction in the frequency and/or severity in ocular lesions within the eye is also one measurement of treatment efficacy of the methods provided herein.

In one embodiment, the suprachoroidal drug dose sufficient to achieve a therapeutic response in a human patient treated with the non-surgical SCS drug delivery method is less than the intravitreal, parenteral, intracameral, topical, or oral drug dose sufficient to elicit the identical or substantially identical therapeutic response. In a further embodiment, the suprachoroidal drug dose is at least 10 percent less than the oral, parenteral or intravitreal dose sufficient to achieve the identical or substantially identical therapeutic response. In a further embodiment, the suprachoroidal dose is about 10 percent to about 25 percent less, or about 10 percent to about 50 percent less than the oral, parenteral, intracameral, topical, or intravitreal dose sufficient to achieve the identical or substantially identical therapeutic response.

In some embodiments, the non-surgical administration of axitinib according to the methods described herein reduces the number and/or frequency of administration of a VEGF modulator to the patient. Thus, in some embodiments, the administration of the axitinib increases the effectiveness and/or durability of the VEGF modulator treatment. For example, in some embodiments, the SCS administration of axitinib results in a need for fewer administrations of a VEGF modulator, and/or results in a longer period of time between administrations of a VEGF modulator.

In some embodiments, the non-surgical administration of axitinib according to the methods described herein results in maintenance of the improved BCVA and/or the improved CST in the patient for at least 12 weeks, at least 16 weeks, at least 20 weeks, at least 24 weeks, at least 30 weeks, at least 36 weeks, at least 40 weeks, at least 44 weeks, at least 48 weeks, or at least 2, 3, 4, 5, 6, 7, 8, or 9 months or longer after the initial dose of axitinib. In some embodiments, the non-surgical administration of axitinib according to the methods described herein results in maintenance of the improved BCVA and/or the improved CST in the patient for at least 12 weeks, at least 16 weeks, at least 20 weeks, at least 24 weeks, at least 30 weeks, at least 36 weeks, at least 40 weeks, at least 44 weeks, at least 48 weeks, at least 52 weeks, or longer after a second dose of axitinib. In some embodiments, the method comprises administering the axitinib to the patient in a first dose and a second dose, wherein the second dose is administered at at about 2 months, at about 3 months, at about 4 months, at about 5 months, or at about 6 months following the first dose. In some embodiments, the method comprises administering the axitinib to the patient in a first dose and a second dose, wherein the second dose is administered about 2 months to about 6 months following the first dose. In some embodiments, the method comprises administering the axitinib to the patient in a first dose and a second dose, wherein the second dose is administered about 4 months following the first dose. In some embodiments, the method comprises administering the axitinib to the patient in a first dose and a second dose, wherein the second dose is administered about 6 months following the first dose. In some embodiments, the method comprises administering the axitinib to the patient in a first dose and a second dose, wherein the second dose is administered about 9 months following the first dose.

In one embodiment, the non-surgical administration of axitinib to the eye according to the methods provided herein results in a decreased number of deleterious side effects or clinical manifestations in the treated patient as compared to the number of side effects or clinical manifestations caused by the same drug dose administered intravitreally, intracamerally, orally or parenterally; or results in a decreased number of deleterious side effects or clinical manifestations in the treated patient as compared to those caused by administration of a drug previously used to treat the disease.

Examples of side effects and clinical manifestations that can be reduced or ameliorated include, but are not limited to, inflammation, gastrointestinal side effects (e.g., diarrhea, nausea, gastroenteritis, vomiting, gastrointestinal, rectal, and duodenal hemorrhage, hemorrhagic pancreatitis, large intestine perforation black or bloody stools, and/or coughing up blood); hematologic side effects (e.g., leucopenia, anemia, pancytopenia and agranulocytosis, thrombocytopenia, neutropenia, pure red cell aplasia (PRCA), deep venous thrombosis easy bruising, and/or unusual bleeding from the nose, mouth, vagina, or rectum); immunologic side effects/clinical manifestations (e.g., immunosuppression, immunosuppression resulting in sepsis, opportunistic infections (herpes simplex virus, herpes zoster, and invasive candidal infections), and/or increased infection); oncologic side effects/clinical manifestations (e.g., lymphoma, lymphoproliferative disease and/or non-melanoma skin carcinoma); renal side effects/clinical manifestations (e.g. dysuria, urgency, urinary tract infections, hematuria, kidney tubular necrosis, and/or BK virus-associated nephropathy); metabolic side effects/clinical manifestations (e.g. edema, hyperphosphatemia, hypokalemia, hyperglycemia, hyperkalemia. swelling, rapid weight gain, and/or enlarged thyroid); respiratory side effects/clinical manifestations (e.g., respiratory infection, dyspnea, increased cough, primary tuberculosis dry cough, wheezing, and/or stuffy nose); dermatologic side effects/clinical manifestations (e.g., acne, rash, dyshidrotic eczema, papulosquamous psoriatic-like skin eruption rash, blisters, oozing, mouth sores, and/or hair loss); muscoskeletal side effects/clinical manifestations (e.g. myopathy and/or muscle pain), hepatic side effects/clinical manifestations (e.g. hepatoxicity and/or jaundice), abdominal pain, increased incidence of first trimester pregnancy loss, missed menstrual periods, severe headache, confusion, change in mental status, vision loss, seizure (convulsions), increased sensitivity to light, dry eye, red eye, itchy eye, and/or high blood pressure.

As provided throughout, the compositions administered herein in one embodiment, the methods described herein comprise administering a tyrosine kinase inhibitor. Exemplary tyrosine kinase inhibitors for use in the methods described herein include, but are not limited to, Alectinib (Alecensa®); angiokinase inhibitors such as Nintedanib (Vargatev®), Afatinib (Gilotrif®), and Motesanib; Apatinib; Axitinib; Cabozantinib (Cometriq®); Canertinib; Crenolanib; Damnacanthal; Foretinib; Fostamatinib; growth factor receptor inhibitor; Ibrutinib (Imbruvica®); Icotinib; Imatinib (Gleevec®); Linifanib; Mubritinib; Radotinib; T790M; V600E; Vatalanib; Vemurafenib (Zelboraf®); AEE788 (TKI, VEGFR-2, EGFR: Novartis); ZD6474 (TKI, VEGFR-1, -2, -3, EGFR: Zactima: AstraZeneca); AZD2171 (TKI, VEGFR-1, -2: AstraZeneca); SU 11248 (TKI, VEGFR-1, -2, PDGFR: Sunitinib: Pfizer); AG13925 (TKI, VEGFR-1, -2: Pfizer); AG013736 (TKI, VEGFR-1, -2: Pfizer); CEP-7055 (TKI, VEGFR-1, -2, -3: Cephalon); CP-547,632 (TKI, VEGFR-1, -2: Pfizer); GW7S6024 (TKL VEGFR-1, -2, -3: GlaxoSmithKline); GW786034 (TKI, VEGFR-1, -2, -3: GlaxoSmithKline); sorafenib (TKI, Bay 43-9006, VEGFR-1, -2, PDGFR: Bayer/Onyx); SU4312 (TKI, VEGFR-2, PDGFR: Pfizer); AMG706 (TKI, VEGFR-1, -2, -3: Amgen); XL647 (TKI, EGFR, HER2, VEGFR, ErbB4: Exelixis); XL999 (TKI, FGFR, VEGFR, PDGFR, FII-3: Exelixis); PKC412 (TKI, KIT, PDGFR, PKC, FLT3, VEGFR-2: Novartis); AEE788 (TKI, EGFR, VEGFR2, VEGFR-1: Novartis): OSI-030 (TKI, c-kil, VEGFR: OSI Pharmaceuticals); OS1-817 (TKI c-kit, VEGFR: OSI Pharmaceuticals); DMPQ (TKI, ERGF, PDGFR, ErbB2. p56. pkA, pkC); MLN518 (TKI, Flt3, PDGFR, c-KIT (T53518: Millennium Pharmaceuticals); lestaurinib (TKI, FLT3, CEP-701, Cephalon); ZD 1839 (TKI, EGFR: gefitinib, Iressa: AstraZeneca); OSI-774 (TKI, EGFR: Erlotininb: Tarceva: OSI Pharmaceuticals); lapatinib (TKI, ErbB-2, EGFR, and GD-2016: Tykerb: GlaxoSmithKline).

Axitinib is a potent tyrosine kinase inhibitor of vascular endothelial growth factor receptors VEGFR-1, VEGFR-2, and VEGFR-3. These receptors are implicated in pathologic angiogenesis, tumor growth, and metastatic progression of cancer. Axitinib has been shown to potently and selectively inhibit VEGF-mediated signaling and endothelial cell proliferation and survival at picomolar concentrations. Axitinib also inhibits other RTKs at low nanomolar concentrations, including PDGFR-α, PDGFR-β, and c-Kit. The most common metabolites of axitinib, the N-glucuronide (M7) and the sulfoxide (M12) were ≥400-fold less potent against VEGFR-2.

“CLS-AX” is used herein to describe a formulation comprising axitinib. Exemplary formulations are provided in Tables 1A and 1B and throughout the disclosure.

In some embodiments, axitinib formulation of the present disclosure comprises axitinib at a concentration of about 1 mg/mL to about 60 mg/mL, for example, about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 21 mg/mL, about 22 mg/mL, about 23 mg/mL, about 24 mg/mL, about 25 mg/mL, about 26 mg/mL, about 27 mg/mL, about 28 mg/mL, about 29 mg/mL, about 30 mg/mL, about 31 mg/mL, about 32 mg/mL, about 33 mg/mL, about 34 mg/mL, about 35 mg/mL, about 36 mg/mL, about 37 mg/mL, about 38 mg/mL, about 39 mg/mL, about 40 mg/mL, about 41 mg/mL, about 42 mg/mL, about 43 mg/mL, about 44 mg/mL, about 45 mg/mL, about 46 mg/mL, about 47 mg/mL, about 48 mg/mL, about 49 mg/mL, about 50 mg/mL, about 51 mg/mL, about 52 mg/mL, about 53 mg/mL, about 54 mg/mL, about 55 mg/mL, about 56 mg/mL, about 57 mg/mL, about 58 mg/mL, about 59 mg/mL, or about 60 mg/mL, including any values or ranges therebetween. In some embodiments, axitinib formulation of the present disclosure comprises axitinib at a concentration of about 40 mg/mL.

In some embodiments, axitinib and/or formulation of the present disclosure has an average particle size D₅₀ of about 0.5 μm to about 10 μm. In some embodiments, axitinib and/or formulation of the present disclosure has a D₅₀ of at least about 1 μm. In some embodiments, axitinib and/or formulation of the present disclosure has a D₅₀ of at least about 2 μm. In some embodiments, axitinib and/or formulation of the present disclosure has a D₅₀ of at least about 5 μm. In some embodiments, axitinib and/or formulation of the present disclosure has a D₅₀ at most about 10 μm. In some embodiments, axitinib and/or formulation of the present disclosure has a D₅₀ of about 0.5 μm to about 1 μm, about 0.5 μm to about 2 μm, about 0.5 μm to about 3 μm, about 0.5 μm to about 4 μm, about 0.5 μm to about 5 μm, about 0.5 μm to about 6 μm, about 0.5 μm to about 7 μm, about 0.5 μm to about 8 μm, about 0.5 μm to about 9 μm, about 0.5 μm to about 10 μm, about 1 μm to about 2 μm, about 1 μm to about 3 μm, about 1 μm to about 4 μm, about 1 μm to about 5 μm, about 1 μm to about 6 μm, about 1 μm to about 7 μm, about 1 μm to about 8 μm, about 1 μm to about 9 μm, about 1 μm to about 10 μm, about 2 μm to about 3 μm, about 2 μm to about 4 μm, about 2 μm to about 5 μm, about 2 μm to about 6 μm, about 2 μm to about 7 μm, about 2 μm to about 8 μm, about 2 μm to about 9 μm, about 2 μm to about 10 μm, about 3 μm to about 4 μm, about 3 μm to about 5 μm, about 3 μm to about 6 μm, about 3 μm to about 7 μm, about 3 μm to about 8 μm, about 3 μm to about 9 μm, about 3 μm to about 10 μm, about 4 μm to about 5 μm, about 4 μm to about 6 μm, about 4 μm to about 7 μm, about 4 μm to about 8 μm, about 4 μm to about 9 μm, about 4 μm to about 10 μm, about 5 μm to about 6 μm, about 5 μm to about 7 μm, about 5 μm to about 8 μm, about 5 μm to about 9 μm, about 5 μm to about 10 μm, about 6 μm to about 7 μm, about 6 μm to about 8 μm, about 6 μm to about 9 μm, about 6 μm to about 10 μm, about 7 μm to about 8 μm, about 7 μm to about 9 μm, about 7 μm to about 10 μm, about 8 μm to about 9 μm, about 8 μm to about 10 μm, or about 9 μm to about 10 μm. In some embodiments, axitinib and/or formulation of the present disclosure has a D₅₀ of about 0.5 μm, about 1 μm, about 2 μm, about 3 μm, about 4 μm, about 5 μm, about 6 μm, about 7 μm, about 8 μm, about 9 μm, or about 10 μm, or including any values or ranges therebetween.

In some embodiments, axitinib formulation comprises one or more pharmaceutically acceptable excipients, for example, axitinib formulation of the present disclosure comprises polysorbate 80 of about 0.01% w/v to about 0.05% w/v. In some embodiments, axitinib formulation comprises polysorbate 80 of at least about 0.01% w/v. In some embodiments, axitinib formulation comprises polysorbate 80 of at most about 0.05% w/v. In some embodiments, axitinib formulation comprises polysorbate 80 of about 0.01% w/v to about 0.015% w/v, about 0.01% w/v to about 0.02% w/v, about 0.01% w/v to about 0.025% w/v, about 0.01% w/v to about 0.03% w/v, about 0.01% w/v to about 0.035% w/v, about 0.01% w/v to about 0.04% w/v, about 0.01% w/v to about 0.045% w/v, about 0.01% w/v to about 0.05% w/v, about 0.015% w/v to about 0.02% w/v, about 0.015% w/v to about 0.025% w/v, about 0.015% w/v to about 0.03% w/v, about 0.015% w/v to about 0.035% w/v, about 0.015% w/v to about 0.04% w/v, about 0.015% w/v to about 0.045% w/v, about 0.015% w/v to about 0.05% w/v, about 0.02% w/v to about 0.025% w/v, about 0.02% w/v to about 0.03% w/v, about 0.02% w/v to about 0.035% w/v, about 0.02% w/v to about 0.04% w/v, about 0.02% w/v to about 0.045% w/v, about 0.02% w/v to about 0.05% w/v, about 0.025% w/v to about 0.03% w/v, about 0.025% w/v to about 0.035% w/v, about 0.025% w/v to about 0.04% w/v, about 0.025% w/v to about 0.045% w/v, about 0.025% w/v to about 0.05% w/v, about 0.03% w/v to about 0.035% w/v, about 0.03% w/v to about 0.04% w/v, about 0.03% w/v to about 0.045% w/v, about 0.03% w/v to about 0.05% w/v, about 0.035% w/v to about 0.04% w/v, about 0.035% w/v to about 0.045% w/v, about 0.035% w/v to about 0.05% w/v, about 0.04% w/v to about 0.045% w/v, about 0.04% w/v to about 0.05% w/v, or about 0.045% w/v to about 0.05% w/v. In some embodiments, axitinib formulation comprises polysorbate 80 of about 0.01% w/v, about 0.015% w/v, about 0.02% w/v, about 0.025% w/v, about 0.03% w/v, about 0.035% w/v, about 0.04% w/v, about 0.045% w/v, or about 0.05% w/v. In some embodiments, axitinib formulation comprises polysorbate 80 of about 0.05% w/v to about 0.1% w/v. In some embodiments, axitinib formulation comprises polysorbate 80 of at least about 0.05% w/v. In some embodiments, axitinib formulation comprises polysorbate 80 of at most about 0.1% w/v. In some embodiments, axitinib formulation comprises polysorbate 80 of about 0.05% w/v to about 0.06% w/v, about 0.05% w/v to about 0.07% w/v, about 0.05% w/v to about 0.08% w/v, about 0.05% w/v to about 0.09% w/v, about 0.05% w/v to about 0.1% w/v, about 0.06% w/v to about 0.07% w/v, about 0.06% w/v to about 0.08% w/v, about 0.06% w/v to about 0.09% w/v, about 0.06% w/v to about 0.1% w/v, about 0.07% w/v to about 0.08% w/v, about 0.07% w/v to about 0.09% w/v, about 0.07% w/v to about 0.1% w/v, about 0.08% w/v to about 0.09% w/v, about 0.08% w/v to about 0.1% w/v, or about 0.09% w/v to about 0.1% w/v. In some embodiments, axitinib formulation comprises polysorbate 80 of about 0.05% w/v, about 0.06% w/v, about 0.07% w/v, about 0.08% w/v, about 0.09% w/v, or about 0.1% w/v.

In some embodiments, axitinib formulation comprises one or more pharmaceutically acceptable excipients, for example, axitinib formulation of the present disclosure comprises polysorbate 80 of about 0.01 wt. % to about 0.05 wt. %. In some embodiments, axitinib formulation comprises polysorbate 80 of at least about 0.01 wt. %. In some embodiments, axitinib formulation comprises polysorbate 80 of at most about 0.05 wt. %. In some embodiments, axitinib formulation comprises polysorbate 80 of about 0.01 wt. % to about 0.015 wt. %, about 0.01 wt. % to about 0.02 wt. %, about 0.01 wt. % to about 0.025 wt. %, about 0.01 wt. % to about 0.03 wt. %, about 0.01 wt. % to about 0.035 wt. %, about 0.01 wt. % to about 0.04 wt. %, about 0.01 wt. % to about 0.045 wt. %, about 0.01 wt. % to about 0.05 wt. %, about 0.015 wt. % to about 0.02 wt. %, about 0.015 wt. % to about 0.025 wt. %, about 0.015 wt. % to about 0.03 wt. %, about 0.015 wt. % to about 0.035 wt. %, about 0.015 wt. % to about 0.04 wt. %, about 0.015 wt. % to about 0.045 wt. %, about 0.015 wt. % to about 0.05 wt. %, about 0.02 wt. % to about 0.025 wt. %, about 0.02 wt. % to about 0.03 wt. %, about 0.02 wt. % to about 0.035 wt. %, about 0.02 wt. % to about 0.04 wt. %, about 0.02 wt. % to about 0.045 wt. %, about 0.02 wt. % to about 0.05 wt. %, about 0.025 wt. % to about 0.03 wt. %, about 0.025 wt. % to about 0.035 wt. %, about 0.025 wt. % to about 0.04 wt. %, about 0.025 wt. % to about 0.045 wt. %, about 0.025 wt. % to about 0.05 wt. %, about 0.03 wt. % to about 0.035 wt. %, about 0.03 wt. % to about 0.04 wt. %, about 0.03 wt. % to about 0.045 wt. %, about 0.03 wt. % to about 0.05 wt. %, about 0.035 wt. % to about 0.04 wt. %, about 0.035 wt. % to about 0.045 wt. %, about 0.035 wt. % to about 0.05 wt. %, about 0.04 wt. % to about 0.045 wt. %, about 0.04 wt. % to about 0.05 wt. %, or about 0.045 wt. % to about 0.05 wt. %. In some embodiments, axitinib formulation comprises polysorbate 80 of about 0.01 wt. %, about 0.015 wt. %, about 0.02 wt. %, about 0.025 wt. %, about 0.03 wt. %, about 0.035 wt. %, about 0.04 wt. %, about 0.045 wt. %, or about 0.05 wt. %. In some embodiments, axitinib formulation comprises polysorbate 80 of about 0.05 wt. % to about 0.1 wt. %. In some embodiments, axitinib formulation comprises polysorbate 80 of at least about 0.05 wt. %. In some embodiments, axitinib formulation comprises polysorbate 80 of at most about 0.1 wt. %. In some embodiments, axitinib formulation comprises polysorbate 80 of about 0.05 wt. % to about 0.06 wt. %, about 0.05 wt. % to about 0.07 wt. %, about 0.05 wt. % to about 0.08 wt. %, about 0.05 wt. % to about 0.09 wt. %, about 0.05 wt. % to about 0.1 wt. %, about 0.06 wt. % to about 0.07 wt. %, about 0.06 wt. % to about 0.08 wt. %, about 0.06 wt. % to about 0.09 wt. %, about 0.06 wt. % to about 0.1 wt. %, about 0.07 wt. % to about 0.08 wt. %, about 0.07 wt. % to about 0.09 wt. %, about 0.07 wt. % to about 0.1 wt. %, about 0.08 wt. % to about 0.09 wt. %, about 0.08 wt. % to about 0.1 wt. %, or about 0.09 wt. % to about 0.1 wt. %. In some embodiments, axitinib formulation comprises polysorbate 80 of about 0.05 wt. %, about 0.06 wt. %, about 0.07 wt. %, about 0.08 wt. %, about 0.09 wt. %, or about 0.1 wt. %.

In some embodiments, the axitinib formulation of the present disclosure comprises about 0.6 wt. % to about 0.9 wt. % of sodium chloride, for example, about 0.61 wt. %, about 0.62 wt. %, about 0.63 wt. %, about 0.64 wt. %, about 0.65 wt. %, about 0.66 wt. %, about 0.67 wt. %, about 0.68 wt. %, about 0.69 wt. %, about 0.70 wt. %, about 0.71 wt. %, about 0.72 wt. %, about 0.73 wt. %, about 0.74 wt. %, about 0.75 wt. %, about 0.76 wt. %, about 0.77 wt. %, about 0.78 wt. %, about 0.79 wt. %, about 0.80 wt. %, about 0.81 wt. %, about 0.82 wt. %, about 0.83 wt. %, about 0.84 wt. %, about 0.85 wt. %, about 0.86 wt. %, about 0.87 wt. %, about 0.88 wt. %, about 0.89 wt. %, about 0.90 wt. %, including any values or ranges therebetween.

In some embodiments, the axitinib formulation of the present disclosure comprises about 0.3 wt. % to about 0.6 wt. % of sodium carboxymethylcellulose, for example, about 0.31 wt. %, about 0.32 wt. %, about 0.33 wt. %, about 0.34 wt. %, about 0.35 wt. %, about 0.36 wt. %, about 0.37 wt. %, about 0.38 wt. %, about 0.39 wt. %, about 0.40 wt. %, about 0.41 wt. %, about 0.42 wt. %, about 0.43 wt. %, about 0.44 wt. %, about 0.45 wt. %, about 0.46 wt. %, about 0.47 wt. %, about 0.48 wt. %, about 0.49 wt. %, about 0.50 wt. %, about 0.51 wt. %, about 0.52 wt. %, about 0.53 wt. %, about 0.54 wt. %, about 0.55 wt. %, about 0.56 wt. %, about 0.57 wt. %, about 0.58 wt. %, about 0.59 wt. %, about 0.60 wt. %, including any values or ranges therebetween.

In some embodiments, the axitinib formulation of the present disclosure comprises about 0.04 wt. % to about 0.08 wt. % of sodium phosphate (monobasic), for example, about 0.041 wt. %, about 0.042 wt. %, about 0.043 wt. %, about 0.044 wt. %, about 0.045 wt. %, about 0.046 wt. %, about 0.047 wt. %, about 0.048 wt. %, about 0.049 wt. %, about 0.05 wt. %, about 0.051 wt. %, about 0.052 wt. %, about 0.053 wt. %, about 0.054 wt. %, about 0.055 wt. %, about 0.056 wt. %, about 0.057 wt. %, about 0.058 wt. %, about 0.059 wt. %, about 0.06 wt. %, about 0.061 wt. %, about 0.062 wt. %, about 0.063 wt. %, about 0.064 wt. %, about 0.065 wt. %, about 0.066 wt. %, about 0.067 wt. %, about 0.068 wt. %, about 0.069 wt. %, about 0.07 wt. %, about 0.071 wt. %, about 0.072 wt. %, about 0.073 wt. %, about 0.074 wt. %, about 0.075 wt. %, about 0.076 wt. %, about 0.077 wt. %, about 0.078 wt. %, about 0.079 wt. %, about 0.08 wt. %, including any values or ranges therebetween.

In some embodiments, the axitinib formulation of the present disclosure comprises about 0.06 wt. % to about 0.10 wt. % of sodium phosphate (dibasic), for example, about 0.061 wt. %, about 0.062 wt. %, about 0.063 wt. %, about 0.064 wt. %, about 0.065 wt. %, about 0.066 wt. %, about 0.067 wt. %, about 0.068 wt. %, about 0.069 wt. %, about 0.07 wt. %, about 0.071 wt. %, about 0.072 wt. %, about 0.073 wt. %, about 0.074 wt. %, about 0.075 wt. %, about 0.076 wt. %, about 0.077 wt. %, about 0.078 wt. %, about 0.079 wt. %, about 0.08 wt. %, about 0.081 wt. %, about 0.082 wt. %, about 0.083 wt. %, about 0.084 wt. %, about 0.085 wt. %, about 0.086 wt. %, about 0.087 wt. %, about 0.088 wt. %, about 0.089 wt. %, about 0.09 wt. %, about 0.091 wt. %, about 0.092 wt. %, about 0.093 wt. %, about 0.094 wt. %, about 0.095 wt. %, about 0.096 wt. %, about 0.097 wt. %, about 0.098 wt. %, about 0.099 wt. %, about 0.1 wt. %, including any values or ranges therebetween.

In some embodiments, the axitinib formulation comprise about 0.01% to about 0.05% polysorbate 80, about 0.5% sodium carboxymethylcellulose, about 0.79% sodium chloride, about 0.06% sodium phosphate monobasic, and about 0.08% sodium phosphate dibasic.

In one embodiment, the tyrosine kinase inhibitor (e.g., axitinib) may be used in combination with one or more agents listed herein or with other agents known in the art, either in a single or multiple formulations. In one embodiment, the agent is a VEGF modulator and is administered intravitreally to the patient in need of treatment. In one embodiment, the VEGF modulator is a VEGF antagonist. In one embodiment, the second drug is a VEGF antagonist including, without limitation, a VEGF-receptor kinase antagonist, an anti-VEGF antibody or fragment thereof, an anti-VEGF receptor antibody, an anti-VEGF aptamer, a small molecule VEGF antagonist, a thiazolidinedione, a quinoline or a designed ankyrin repeat protein (DARPin). In some embodiments, the method further comprises administering a VEGF antagonist to the patient. In some embodiments, the VEGF antagonist includes, but is not limited to, aflibercept, ziv-aflibercept, faricimab (i.e., RG7716), bevacizumab, sonepcizumab, VEGF sticky trap, cabozantinib, foretinib, vandetanib, nintedanib, regorafenib, cediranib, ranibizumab, lapatinib, sunitinib, sorafenib, plitidepsin, regorafenib, verteporfin, bucillamine, axitinib, pazopanib, fluocinolone acetonide, nintedanib, AL8326, 2C3 antibody, AT001 antibody, XtendVEGF antibody, HuMax-VEGF antibody, R3 antibody, AT001/r84 antibody, HyBEV, ANG3070, APX003 antibody, APX004 antibody, ponatinib, BDM-E, VGX100 antibody, VGX200, VGX300, COSMIX, DLX903/1008 antibody, ENMD2076, INDUS815C, R84 antibody, KD019, NM3, MGCD265, MG516, MP0260, NT503, anti-DLL4/VEGF bispecific antibody, PAN90806, Palomid 529, BD0801 antibody, XV615, lucitanib, motesanib diphosphate, AAV2-sFLT01, soluble Flt1 receptor, AV-951, Volasertib, CEP11981, KH903, lenvatinib, lenvatinib mesylate, terameprocol, PF00337210, PRS050, SP01, carboxyamidotriazole orotate, hydroxychloroquine, linifanib, ALG1001, AGN150998, MP0112, AMG386, ponatinib, PD173074, AVA101, BMS690514, KH902, golvatinib (E7050), dovitinib, dovitinib lactate (TKI258, CHIR258), ORA101, ORA102, Axitinib (Inlyta, AG013736), PTC299, pegaptanib sodium, troponin, EG3306, vatalanib, Bmab100, GSK2136773, Anti-VEGFR Alterase, Avila, CEP7055, CLT009, ESBA903, GW654652, HMPL010, GEM220, HYB676, JNJ17029259, TAK593, Nova21012, Nova21013, CP564959, smart Anti-VEGF antibody, AG028262, AG13958, CVX241, SU14813, PRS055, PG501, PG545, PTI101, TG100948, ICS283, XL647, enzastaurin hydrochloride, BC194, COT601M06.1, COT604M06.2, MabionVEGF, Apatinib, RAF265 (CHIR-265), Motesanib Diphosphate (AMG-706), Lenvatinib (E7080), TSU-68 (SU6668, Orantinib), Brivanib (BMS-540215), MGCD-265, AEE788 (NVP-AEE788), ENMD-2076, OSI-930, CYC116, Ki8751, Telatinib, KRN 633, SAR131675, Dovitinib (TKI-258) Dilactic Acid, Apatinib, BMS-794833, Brivanib Alaninate (BMS-582664), Golvatinib (E7050), Semaxanib (SU5416), ZM 323881 HCl, Cabozantinib malate (XL184), ZM 306416, AL3818, AL8326, 2C3 antibody, AT001 antibody, HyBEV, bevacizumab (Avastin®), ANG3070, APX003 antibody, APX004 antibody, ponatinib (AP24534), BDM-E, VGX100 antibody (VGX100 CIRCADIAN), VGX200 (c-fos induced growth factor monoclonal antibody), VGX300, COSMIX, DLX903/1008 antibody, ENMD2076, sunitinib malate (Sutent®), INDUS815C, R84 antibody, KD019, NM3, allogenic mesenchymal precursor cells combined with an anti-VEGF antagonist (e.g., anti-VEGF antibody), MGCD265, MG516, VEGF-Receptor kinase inhibitor, MP0260, NT503, anti-DLL4/VEGF bispecific antibody, PAN90806, Palomid 529, BD0801 antibody, XV615, lucitanib (AL3810, E3810), AMG706 (motesanib diphosphate), AAV2-sFLT01, soluble Flt1 receptor, cediranib (Recentin™), AV-951, tivozanib (KRN-951), regorafenib (Stivarga®), volasertib (BI6727), CEP11981, KH903, lenvatinib (E7080), lenvatinib mesylate, terameprocol (EM1421), ranibizumab (Lucentis®), pazopanib hydrochloride (Votrient™), PF00337210, PRS050, SP01 (curcumin), carboxyamidotriazole orotate, hydroxychloroquine, linifanib (ABT869, RG3635), fluocinolone acetonide (Iluvien®), ALG1001, AGN150998, DARPin MP0112, AMG386, ponatinib (AP24534), AVA101, nintedanib (Vargatef™), BMS690514, KH902, golvatinib (E7050), everolimus (Afinitor®), dovitinib lactate (TKI258, CHIR258), ORA101, ORA102, axitinib (Inlyta®, AG013736), plitidepsin (Aplidin®), PTC299, aflibercept (Zaltrap®, Eylea®), pegaptanib sodium (Macugen™, LI900015), verteporfin (Visudyne®), bucillamine (Rimatil, Lamin, Brimani, Lamit, Boomiq), R3 antibody, AT001/r84 antibody, troponin (BLS0597), EG3306, vatalanib (PTK787), Bmab100, GSK2136773, Anti-VEGFR Alterase, Avila, CEP7055, CLT009, ESBA903, HuMax-VEGF antibody, GW654652, HMPL010, GEM220, HYB676, JNJ17029259, TAK593, XtendVEGF antibody, Nova21012, Nova21013, CP564959, Smart Anti-VEGF antibody, AG028262, AG13958, CVX241, SU14813, PRS055, PG501, PG545, PTI101, TG100948, ICS283, XL647, enzastaurin hydrochloride (LY317615), BC194, quinolines, COT601M06.1, COT604M06.2, Mabion VEGF, SIR-Spheres coupled to anti-VEGF or VEGF-R antibody, Apatinib (YN968D1), or AL3818.

### Treatment Methods

In some embodiments, the compositions and methods provided herein are for use in treating ocular disease and disorders. Exemplary ocular diseases and disorders include, without limitation, wet AMD, nonexudative AMD, CNV, RVO (including central RVO, hemi-RVO, branch RVO), retinopathy, diabetic retinopathy, and diabetic macular edema (DME). In some embodiments, the disease or disorder is macular edema (ME). ME may occur in association with and/or due to central RVO, hemiretinal RVO, branch RVO, inflammation, uveitis, or CNV. In some embodiments, the disease or disorder is a geographic atrophy, for example, from AMD, degenerative retinal disorders, or hereditary retinal disorders. In some embodiments, the disease or disorder is retinal neovascularization. For example, in some embodiments, retinal neovascularization can result form ischemic causes such as diabetic retinopathy, central RVO, hemiretinal RVO, branch RVO, central retinal artery occlusion, branch retinal artery occlusion, sickle cell retinopathy, or retinopathy of prematurity. In embodiment, retinal neovascularization can result form inflammatory and uveitic disorders.

Particular conditions in which choroidal neovascularization may occur include wet AMD, angloid streaks, anterior ischemic optic neuropathy, bacterial endocarditis, Best disease, birdshot retinochroidopathy, choroidal hemanioma, chorodial nevi, choroidal nonperfusion, choroidal osteomas, choroidal rupture, choroidermia, chronic retinal detachment, coloboma of the retina, diabetes mellitus, drusen, endogenous candida endophthalmitis, extrapapillary hematomas of the retinal pigment epithelium, fundus flavimaculatus, an idiopathic condition, macular hole, malignant melanoma, membranoproliferative glomerulonephritis (type II), metallic intraocular foreign body, morning-glory disc syndrome, retinitis pigmentosa, retinochoroidal coloboma, Rubella, sarcoidosis, serpiginous or geographic choroiditis, subretinal fluid drainage, tilted disc syndrome, toxoplasma retinochoroiditis, tuberculosis, Vogt-Koyanagi-Harada syndrome, idiopathic polypoidal choroidal vasculopathy, ocular ischemic syndrome, and carotid stenosis.

In some embodiments, the present disclosure provides a method of treating neovascular age-related macular degeneration (wet AMD), choroidal neovascularization (CNV), or wet AMD associated with CNV comprising, non-surgically administering an effective amount of a formulation comprising axitinib to the suprachoroidal space (SCS) of the eye of a human patient in need thereof.

In some embodiments, the effective amount of axitinib is a dose of about 0.2 mg to 3 mg of axitinib, for example, about 0.2 mg, about 0.3 mg, about 0.4 mg, about 0.5 mg, about 0.6 mg, about 0.7 mg, about 0.8 mg, about 0.9 mg, about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, about 2.0 mg, about 2.1 mg, about 2.2 mg, about 2.3 mg, about 2.4 mg, about 2.5 mg, about 2.6 mg, about 2.7 mg, about 2.8 mg, about 2.9 mg, about 3.0 mg, about 3.2 mg, about 3.4 mg, about 3.6 mg, about 3.8 mg, about 4.0 mg, about 4.2 mg, about 4.4 mg, about 4.6 mg, about 4.8 mg, about 5.0 mg, about 5.2 mg, about 5.4 mg, about 5.6 mg, about 5.8 mg, about 6.0 mg, about 0.3 mg to about 3 mg, about 0.3 mg to about 2 mg, about 0.3 mg to about 1 mg, about 0.5 mg to about 3 mg, about 0.5 mg to about 2 mg, about 0.5 mg to about 1 mg, or about 1 mg to about 2 mg, including all values and ranges therebetween. In some embodiments, the effective amount of axitinib in the formulation is about 0.5 mg. In some embodiments, the effective amount of axitinib in the formulation is about 1.0 mg. In some embodiments, the present disclosure provides a method of treating neovascular age-related macular degeneration (wet AMD), choroidal neovascularization (CNV), or wet AMD associated with CNV comprising non-surgically administering an effective amount of a formulation comprising axitinib to the suprachoroidal space (SCS) of the eye of a human patient in need thereof and the effective amount of axitinib in the formulation is about 0.5 mg to about 1.0 mg.

In some embodiments, the method comprises administering the axitinib to the patient in a first dose and a second dose. In some embodiments, the method further comprises administering a first dose, a second dose, and a third dose. In some embodiments, the method further comprises administering a first dose, a second dose, a third dose, and a fourth dose.

In some embodiments, axitinib is administered in a first and a second dose, wherein the second dose is administered about 2 months to about 12 months following the first dose (e.g., about 2 months to about 11 months, about 3 months to about 11 months, about 3 months to about 10 months, about 3 months to about 9 months, about 3 months to about 8 months, about 3 months to about 7 months, about 3 months to about 6 months, about 2 months, about 3 months, about 4 months, about 5 months, about 6 months, about 7 months, about 8 months, about 9 months, about 10 months, about 11 months, about 12 months or any ranges or values therebetween). In some embodiments, axitinib is administered in a first and a second dose, wherein the second dose is administered about 3 months to about 6 months following the first dose. In some embodiments, the second dose is administered at about 3 months, at about 4 months, at about 5 months, or at about 6 months, following the first dose.

In some embodiments, the formulation comprising the effective amount of axitinib at 1.0 mg is administered in a first dose. In some embodiments, the method further comprises administering one or more maintenance doses. In some embodiments, at least one of the one or more maintenance doses is administered about 2 months to about 12 months (e.g., about 2 months to about 12 months, about 3 months to about 11 months, about 3 months to about 10 months, about 3 months to about 9 months, about 3 months to about 8 months, about 3 months to about 7 months, about 3 months to about 6 months, about 3 months, about 4 months, about 5 months, about 6 months, or any ranges or values therebetween) following the first dose. In some embodiments, wherein at least one of the one or more maintenance doses is administered about 2 months to about 8 months following the first dose. In some embodiments, wherein at least one of the one or more maintenance doses is administered about 3 months to about 6 months following the first dose. In some embodiments, wherein at least one of the one or more maintenance doses is administered at about 3 months, at about 4 months, at about 5 months, or at about 6 months, following the first dose.

In some embodiments, wherein at least one of the one or more maintenance doses is administered about every 4-36 weeks following the first dose (e.g., about 4 weeks to about 36 weeks, about 4 weeks to about 32 weeks, about 4 weeks to about 28 weeks, about 4 weeks to about 24 weeks, about 4 weeks to about 12 weeks, about 6 weeks to about 36 weeks, about 6 weeks to about 32 weeks, about 6 weeks to about 28 weeks, about 6 weeks to about 24 weeks, about 6 weeks to about 12 weeks, about 8 weeks to about 36 weeks, about 8 weeks to about 32 weeks, about 8 weeks to about 28 weeks, about 8 weeks to about 24 weeks, about 8 weeks to about 12 weeks, about 12 weeks to about 36 weeks, about 12 weeks to about 32 weeks, about 12 weeks to about 28 weeks, about 12 weeks to about 24 weeks, about 24 weeks to about 36 weeks, about 24 weeks to about 32 weeks, about 24 weeks to about 28 weeks, or any ranges or values therebetween). In some embodiments, wherein at least one of the one or more maintenance doses is administered about every 6-32 weeks following the first dose. In some embodiments, wherein at least one of the one or more maintenance doses is administered about every 8-28 weeks following the first dose. In some embodiments, wherein at least one of the one or more maintenance doses is administered about every 12-24 weeks following the first dose. In some embodiments, at least one of the one or more maintenance doses is administered about every 24 weeks.

In some embodiments, the treatment reduces the injection frequency for the human patient within about 24 weeks to about 36 weeks following the first dose compared to a human patient who is not administered the formulation comprising the effective amount of axitinib to the SCS. In some embodiments, the method reduces injection frequency for the human patient within about 24 weeks to about 36 weeks following the first dose compared to the injection frequency of the human patient prior to administration of the formulation. In some embodiments, the injection frequency is reduced to about 30-90% fewer doses (e.g., about 30%, about 35%, about 40%, about 45%, about 50%, about 55%, about 60%, about 65%, about 70%, about 75%, about 80%, about 85%, or about 90% fewer doses, including any values or ranges therebetween) within about 24 weeks to about 36 weeks following the first dose. In some embodiments, the injection frequency is reduced to about 1-4 fewer injections (e.g., 1, 2, 3, or 4 fewer injections, including any values or ranges therebetween) within about 24 weeks to about 36 weeks following the first dose.

In some embodiments, the human patient does not require a maintenance dose within about 3 months, 4 months, or 6 months following the first dose, determined by Disease Activity Assessment (DAA). In some embodiments, the human patient requires only one maintenance dose within about 3 months, 4 months, or 6 months following the first dose, determined by Disease Activity Assessment (DAA). In some embodiments, the human patient requires only two maintenance doses within about 3 months, 4 months, or 6 months following the first dose, determined by Disease Activity Assessment (DAA). In some embodiments, DAA criteria include: i) decrease in BCVA (reduction of >10 letters from baseline); ii) increase in CST (increase of >100 microns on SD-OCT from baseline); iii) BCVA reduction of <5 letters from baseline and increase in CST of >75 microns from baseline; and/or iv) new or worsening vision-threatening hemorrhage due to wet AMD.

In some embodiments, the present disclosure provides a method of treating neovascular age-related macular degeneration (wet AMD), choroidal neovascularization (CNV), or wet AMD associated with CNV comprising, non-surgically administering an effective amount of a formulation comprising axitinib to the suprachoroidal space (SCS) of the eye of a human patient in need thereof, wherein the effective amount of axitinib in the formulation is about 0.5 mg to about 1.0 mg, and wherein the human patient has been previously treated for wet AMD or wet AMD associated choroidal neovascularization (CNV). In some embodiments, the human patient has been previously treated with a VEGF antagonist. In some embodiments, the VEGF antagonist is aflibercept, faricimab, bevacizumab, ranibizumab, or pegaptinib sodium. In some embodiments, the VEGF antagonist is aflibercept. In some embodiments, the aflibercept is administered at a dose of about 1 mg to about 3 mg, about 1 mg to about 5 mg, about 1 mg to about 10 mg, about 5 mg to about 7 mg, including any values or ranges therebetween. In some embodiments, the aflibercept is administered at a dose of about 1 mg to about 3 mg. In some embodiments, the aflibercept is administered at a dose of about 2 mg. In some embodiments, the VEGF antagonist is faricimab. In some embodiments, the faricimab is administered at a dose of about 5 mg to about 7 mg, e.g., about 5 mg, about 6 mg, or about 7 mg, including any values or ranges therebetween. In some embodiments, the faricimab is administered at a dose of about 6 mg. In some embodiments, the aflibercept is administered at a dose of about 2 mg. In some embodiments, the VEGF antagonist is administered via intravitreal injection.

In some embodiments, the VEGF antagonist has been administered for about 2 to 4 times within about 4 to about 6 months prior to administration of the formulation. In some embodiments, the treatment method as described herein reduces the injection frequency within about 24 weeks to about 36 weeks following the first dose compared to a comparable method of administering 2 mg of aflibercept. In some embodiments, the injection frequency is reduced to about 30-90% fewer doses (e.g., about 30%, about 35%, about 40%, about 45%, about 50%, about 55%, about 60%, about 65%, about 70%, about 75%, about 80%, about 85%, or about 90% fewer doses, including any values or ranges therebetween) compared to a comparable method of administering 2 mg of aflibercept (e.g., intravitreal administration of aflibercept). In some embodiments, the injection frequency is reduced to at least 50% fewer doses compared to a comparable method of administering 2 mg of aflibercept (e.g., intravitreal administration of aflibercept). In some embodiments, the injection frequency is reduced to about 1-4 fewer injections (e.g., 1, 2, 3, or 4 fewer injections, including any values or ranges therebetween) compared to a comparable method of administering 2 mg of aflibercept (e.g., intravitreal administration of aflibercept). In some embodiments, the injection frequency is reduced to at least 2 fewer injections compared to a comparable method of administering 2 mg of aflibercept (e.g., intravitreal administration of aflibercept).

In some embodiments, the patient does not require additional therapy following about 1 month, about 2 months, about 3 months, about 4 months, about 5 months, or 6 months, including any values or ranges therebetween, following the last administration of the formulation. In some embodiments, the human patient does not require an additional VEGF therapy following about 3 months, 4 months, or 6 months following the last administration of the formulation, determined by Disease Activity Assessment (DAA). In some embodiments, the additional therapy is intravitreal aflibercept or intravitreal faricimab treatment. In some embodiments, the axitinib formulation comprises about 1 mg/ml axitinib. In some embodiments, the VEGF antagonist has been administered for about 1 to 10 times, 1 to 8 times, 1 to 6 times, 1 to 4 times, or 2 to 4 times within about 1 to 10 months, about 2 to 8 months, about 4 to about 6 months, including any values or ranges therebetween, prior to administration of the formulation.

In some embodiments, the methods described herein achieves at least about 20%, at least about 30%, at least about 40%, at least about 50%, at least about 60%, at least about 70%, at least about 80%, at least about 90%, or about 100% reduction in treatment burden (as measured by total injections over treatment period). In some embodiments, the method achieves about 60% to 90%, about 65% to 90%, about 70% to 90%, about 75% to 90%, about 75% to 85%, including any values or ranges therebetween, reduction in treatment burden. In some embodiments, the methods described herein achieves at least about 20%, at least about 30%, at least about 40%, at least about 50%, at least about 60%, at least about 70%, at least about 80%, at least about 90%, or about 100% reduction in treatment burden for 1, 2, 3, 4, 5, 6, 7, 8, 9, 10, 11, 12 months or longer following treatment.

### EXAMPLES

The present invention is further illustrated by reference to the following Examples. However, it should be noted that these Examples, like the embodiments described above, are illustrative and are not to be construed as restricting the scope of the invention in any way.

### Example 1. Axitinib Formulation

One exemplary Axitinib formulation, denoted CLS-AX, includes the following components.

TABLE 1A
Exemplary Axitinib Formulation

| | CLS-AX |
|---|---|
| Ingredient | (% w/v or as noted) |
| Axitinib | 40 mg/mL |
| Carboxymethylcellulose Sodium (NaCMC) | 0.5% |
| Polysorbate 80 (PS-80) | 0.1% (or 0.01% to 0.05%) |
| Sodium chloride | 0.79% |
| Sodium phosphate, monobasic, monohydrate | 0.059% |
| Sodium phosphate, dibasic, anhydrous | 0.079% |
| Water for Injection | q.s. |
| Sodium hydroxide/Hydrochloric acid | Adjust pH to 6.8 |

In exemplary axitinib formulations, the axitinib is present at a concentration of about 1 mg/mL to about 40 mg/mL. In addition, in some exemplary axitinib formulations, the polysorbate 80 is present in the formulation at about 0.04% w/v to about 0.05% w/v. In some exemplary axitinib formulations, the polysorbate 80 is present in the formulation at about 0.01% w/v to about 0.05% w/v. Further, in some exemplary embodiments, the sodium chloride is present at a concentration of 0.8%, and/or the sodium phosphate (monobasic, monohydrate) is present at 0.05% w/v; and/or the sodium phosphate (dibasic, anhydrous) is present at 0.085% w/v. Accordingly, the polysorbate 80 concentration may range from about 0.01% w/v to about 0.1% w/v, the sodium chloride concentration may range from about 0.7% w/v to about 0.9% w/v, the sodium phosphate (monobasic, monohydrate) concentration may range from about 0.05% to about 0.06% w/v, and the sodium phosphate (dibasic, anhydrous) may range from about 0.075% w/v to about 0.085% w/v.

One exemplary axitinib formulation, also denoted in the table below as CLS-AX, is a 1 mg/mL formulation and includes the following components.

TABLE 1B
Exemplary Axitinib Formulation

| | Composition | |
|---|---|---|
| Component | (% w/w) | Quantity per Batch (5029 g) |
| Axitinib | 0.0994 | 5.00 g + 0.05 g |
| Super Refined Tween | 0.050 | 2.51 g + 0.05 g |
| 80-LQ-(MH), | | |
| Polysorbate 80 | | |
| 7 MF PH Sodium | 0.497 | 30.00 g + 0.30 g |
| Carboxymethylcellulose | | |
| Sodium Chloride, | 0.794 | 47.92 g + 0.48 g |
| Powder | | |
| Sodium Phosphate, | 0.060 | 3.62 g + 0.04 g |
| Monobasic, | | |
| Monohydrate | | |
| Sodium Phosphate, | 0.080 | 4.83 g + 0.05 g |
| Dibasic, Anhydrous, | | |
| Extra Pure | | |
| Water for Injection | 98.4194 | q.s. to |
| (WFI) | | 5,029.0 g + 1% |

In some embodiments, the NaCMC or a similar compound is included in the formulation as a viscosity modifier. In some embodiments, the polysorbate 80 (PS-80) or a similar agent is included in the formulation as a surfactant wetting agent for the active pharmaceutical ingredient, axitinib. In some embodiments, the sodium chloride is included in the formulation as a tonicity adjuster. In some embodiments, the sodium phosphate, monobasic, monohydrate; and/or the sodium phosphate, dibasic, anhydrous are included as pH buffers. In some embodiments, the sodium hydroxide/hydrochloric acid is included in the formulation as a pH adjuster. In some embodiments, the water for injection is the solvent of the formulation.

In some embodiments, no preservative is present. In some embodiments, the formulation is terminally sterilized via autoclave.

Further information providing the solubility, particle size, viscosity, and other product profile parameters of exemplary CLS-AX compositions (active pharmaceutical ingredient (API); and drug product) are provided in Table 1C.

TABLE 1C
Exemplary Product Profile

| | | |
|---|---|---|
| API | Chemical formula | C22H18N4OS |
| | Molecular weight | 386.47 |
| | (g/mol) | |
| | Melting point (° C.) | 213-215 |
| | Boiling Point (° C.) | 668.9 ± 55.0 |
| | Density (g/mL) | 1.4 |
| | Solubility | DMSO |
| | | 0.2 μg/mL in water |
| | Particle size (±2) | D50 - 2 μm |
| | | D90 - 3 μm |

| | | Preclinical | Clinical |
|---|---|---|---|
| Drug | Particle size (±2) | D50 - 1 μm | D10 - 2 μm |
| Product | | D90 - 3 μm | D50 - 5 μm |
| | | | D90 - 10 μm |

| | |
|---|---|
| Viscosity (cP) | 6 |

### Example 2. Safety and Tolerability Study of Suprachoroidal Injection of CLS-AX Following Anti-VEGF Therapy in Neovascular AMD

A clinical study was performed to assess the safety and efficacy of non-surgical administration of axitinib to the suprachoroidal space of the eye of wet AMD patients. In part, the tested doses were in line with the data obtained in the pharmacodynamics studies disclosed herein.

The primary objective of this study was to assess safety and tolerability of CLS-AX in subjects with neovascular age-related macular degeneration who show stable visual acuity following 3 or more injections with an intravitreal anti-VEGF therapy in the preceding 5 months by monitoring the following:
Incidence of treatment-emergent adverse events (TEAEs) from Day 1 to Week 12;
Incidence of serious adverse events from Day 1 to Week 12.

The secondary objective of this study was to evaluate and compare the effect of 4 cohort regimens of CLS-AX over 3 months on visual function and anatomy and the need for additional treatment with intravitreal aflibercept.

Study Design: This was a open-label, dose-escalation study to assess the safety and tolerability of single doses of CLS-AX administered suprachoroidally following at least 3 prior treatments (the last of which was administered at the Screening visit) with an intravitreal (IVT) anti-VEGF agent in wet AMD subjects. The study design included 4 dose cohorts. Subject eligibility was established at Visit 1, Screening (Day −28±3 days). Eligible subjects received an IVT injection of aflibercept, 2 mg (0.05 mL), at Visit 1, Screening (Day −28±3 days), followed by a suprachoroidal injection of CLS-AX at Visit 2, Baseline (Day 0). Subjects returned for safety and tolerability assessments, visual function and ocular anatomy assessments, and the need for additional treatment at Visits 3, 4, and 5 (Weeks 4, 8 and 12), (Follow-up Period). Further, additional treatments were administered at Visits 3 and 4 (Weeks 4 and 8) based on PRN criteria and were consist of aflibercept (2 mg (0.05 mL) administered by IVT injection. All subjects were followed until Visit 5 (Week 12, Study Exit) regardless of whether additional therapy was given or not. The 4 dose cohorts included the following:
Cohort 1 (lowest dose arm): 0.03 mg/100 μL suprachoroidal injection of CLS-AX
Cohort 2 (mid dose arm): 0.10 mg/100 μL suprachoroidal injection of CLS-AX
Cohort 3 (high-mid dose arm): 0.50 mg/100 μL suprachoroidal injection of CLS-AX
Cohort 4 (high dose arm): 1.0 mg/100 μL suprachoroidal injection of CLS-AX

All cohorts were assessed for safety and tolerability and effects on visual function and anatomy as outlined in the time and events schedule.

Inclusion criteria: Subjects were eligible for participation in this study if s/he met all of the following criteria at the Screening visit (Visit 1) and Baseline visit (Visit 2):
1. Diagnosis of neovascular age-related macular degeneration in the study eye
2. Active subfoveal choroidal neovascularization (CNV) secondary to AMD of any lesion type in the study eye with photos and/or fluorescein angiography (FA) and/or spectral-domain optical coherence tomography (SD-OCD) showing: a. the total lesion area (including blood, neovascularization and scar/atrophy)≤30 mm², b. CNV component area of ≥50% of total lesion area, c. CNV must not be associated with subfoveal hemorrhage, subfoveal fibrosis or subfoveal atrophy.
3. At Screening, two or more anti-VEGF treatments (aflibercept, ranibizumab, bevacizumab, brolucizumab) in the 4 months preceding Screening (Visit 1), with the last treatment administered at least 4 weeks prior to Screening (Visit 1), for neovascular AMD in the study eye with a meaningful response, for example, an improvement in vision and/or exudation, based on the Investigator's opinion.
4. Early Treatment Diabetic Retinopathy Study (ETDRS) best-corrected visual activity (BCVA) score of ≥20 letters read (20/400 Snellen equivalent) and ≤75 letters read (20/32 Snellen equivalent) in the study eye with less than 5 letters change in BCVA between Visit 1 and Visit 2 (Screening and Baseline)
5. Understands the language of the informed consent; willing and able to provide written informed consent prior to any study procedures; willing to comply with the instructions and attend all scheduled study visits
6. At least 50 years of age
   Ophthalmic Exclusion criteria: Subjects were ineligible for participation in this study if s/he met any of the following criteria:
1. Any active ocular disease or infection in the study eye other than wet AMD, such as central serous retinopathy, polypoidal choroidal vasculopathy (PCV), macular edema with etiology other than wet AMD, diabetic retinopathy, and/or current or prior evidence of toxic maculopathy.
2. CNV in the study eye due to other causes other than AMD, such as pathologic myopia, ocular histoplasmosis, angioid streaks, choroidal rupture, and/or uveitis.
3. CNV in the study eye with any of the following on photos and/or FA and/or SD-OCT: total lesion area >30 mm², CNV component area of <50% of total lesion area, subfoveal hemorrhage, subfoveal fibrosis or subfoveal atrophy, retinal pigment epithelial tear, and/or retinal angiomatous proliferation (RAP).
4. Other than IVT injected aflibercept, ranibizumab, bevacizumab, or brolucizumab, prior treatment for CNV in the study eye, including laser photocoagulation; verteporfin photodynamic therapy; intravitreal or periocular corticosteroids; intravitreal gas or air; tissue plasminogen activator; transpupillary thermotherapy; ocriplasmin; and/or pars plana vitrectomy.
5. Has significant media opacity in the study eye precluding evaluation of the retina and Vitreous.
6. Intraocular pressure >25 mmHg in the study eye; subjects are not excluded if intraocular pressure (IOP) is <25 mmHg in the study eye with no more than 1 IOP lowering medications; or cup-to-disc ratio >0.8.
7. Any concurrent condition in the study eye that, in the opinion of the Investigator, could reduce the potential for visual improvement or require medical or surgical intervention, such as amblyopia, aphakia, cataract of visual significance, epiretinal membrane, with traction, intraocular inflammation or uveitis, and/or vitreous hemorrhage.
8. Any prior ocular surgery in the study eye, such as:
a. Panretinal or macular laser photocoagulation, laser photocoagulation, cryotherapy;
b. Vitreoretinal surgery (scleral buckle, pars plana vitrectomy, retrieval of a dropped nucleus or intraocular lens);
c. Glaucoma laser procedures or surgery (cyclodestructive procedures or filtration surgeries, minimally invasive glaucoma surgery);
d. Corneal transplantation;
e. Cataract surgery or Yttrium-Aluminum-Garnet (YAG) laser capsulotomy within 3 months of Screening (Visit 1); and/or
f. Refractive procedures within 3 months of Screening (Visit 1).
9. Has high myopia in the study eye defined as a spherical equivalent >−6 diopters or an axial length ≥26 mm. Has a prior history of high myopia, if pseudophakic.
10. In the study eye, any topical ocular corticosteroid in the 10 days prior to treatment at Baseline (Visit 2); any intraocular or periocular corticosteroid injection.
11. Concomitant therapy with any drug that may be toxic to the lens, retina or optic nerve, such as (examples for clarification purposes; list is not exhaustive): hydroxychloroquine or chloroquine.
   General Exclusion Criteria: Subjects were Ineligible for Participation in this Study if s/he Met any of the Following Criteria:
12. Female subjects who were pregnant, lactating or planning a pregnancy. Females of childbearing potential must agree to submit to a pregnancy test at screening and agree to use an acceptable method of contraception throughout participation in the study. Acceptable methods of contraception include double barrier methods (condom with spermicide or diaphragm with spermicide), hormonal methods (oral contraceptives, implantable, transdermal, or injectable contraceptives), or an intrauterine contraceptive device with a documented failure rate of less than 1% per year.
13. Any uncontrolled systemic disease that, in the opinion of the Investigator, would preclude participation in the study (e.g., unstable medical status including uncontrolled elevated blood pressure, cardiovascular disease, and glycemic control) or put the subject at risk due to study treatment or procedures.
14. Had taken systemic corticosteroids at doses greater than 20 mg per day for oral prednisone (or equivalent for other corticosteroids) in the 2 weeks prior to Visit 2; subjects on 20 mg or less per day can be enrolled if no increase in dosing was anticipated for the duration of the study.
15. Likely needed for hospitalization or surgery within the study period.
16. Hypersensitivity to any component of the CLS-AX, fluorescein, or to topical anesthetics.
17. Enrolled in an investigational drug or device study or has used an investigational drug or device within 30 days of Visit 1.
   Investigational product, dosage and mode of administration: CLS-AX, axitinib injectable suspension as described in Example 1, doses at 0.03 mg, 0.1 mg, 0.5 mg, and 1.0 mg in 100 μL into the suprachoroidal space, were administered with the SCS Microinjector.
   Primary endpoint: The primary endpoint for evaluating the tolerability of the four dose cohorts was based on the incidence of treatment-emergent adverse events (TEAEs), and serious adverse events (SAEs). TEAEs were defined as an event that emerged during treatment with CLS-AX that was either absent pre-treatment or worsened relative to the pre-treatment state.
   Secondary endpoints: Secondary endpoints include:
Incidence/descriptive statistics of changes in safety parameters including: intraocular pressure (IOP), slit-lamp biomicroscopy outcomes, indirect ophthalmoscopy outcomes, and imaging parameters in the study eye; and laboratory evaluations, 12-lead electrocardiograms, and vital signs at Visits 3, 4 and 5 (Weeks 4, 8 and 12)
Number and percentage of participants receiving additional IVT aflibercept injections in the study eye at Visits 3 and 4 (Weeks 4 and 8)
Number and percentage of participants meeting the additional IVT aflibercept therapy criteria in the study eye at Visits 3, 4 and 5 (Weeks 4, 8 and 12)
Mean change from Baseline (Visit 2) in central subfield retinal thickness (CST) in the study eye at Visits 3, 4 and 5 (Weeks 4, 8 and 12)
Mean change from Baseline (Visit 2) in BCVA in the study eye at Visits 3, 4 and 5 (Weeks 4, 8 and 12).
   Additional therapy criteria: At Visit 3 (Week 4), Visit 4 (Week 8), and Visit 5 (Week 12) participants were evaluated for the need for additional therapy for wet AMD in the study eye based on the following criteria. Additional treatment was to be aflibercept, 2 mg (0.05 mL) administered by IVT injection unless other a different therapy was medically necessary; even if additional therapy was given, the participant was to remain in the study and followed until Visit 5 (Study Exit). If any of the following criteria were met in the study eye at Visit 3 (Week 4), or Visit 4 (Week 8), then aflibercept was to be administered:
Loss of 10 or more letters in BCVA compared to the best prior study-assessed BCVA in the study eye that was attributed to intra- or sub-retinal fluid observed by the Investigator, OR
Increase in central subfield retinal thickness greater than 75 microns from Visit 2 (Baseline) in the study eye, OR
Presence of vision-threatening hemorrhage due to AMD in the study eye.

### Safety and Tolerability Results

In all cohorts in the 3-month study (n=27), no serious adverse events (SAEs), no treatment emergent adverse events (TEAEs) related to study treatment, and no dose limiting toxicities were observed. No adverse events related to inflammation, vasculitis or vascular occlusion were observed. No vitreous “floaters” or dispersion of CLS-AX into the vitreous was observed. No retinal detachments, endophthalmitis, or adverse events related to intraocular pressure were observed.

### Example 3. Extension Study to Evaluate the Long-Term Outcomes of Subjects Following CLS-AX Administration for Age-Related Macular Degeneration

This was an open-label, non-interventional extension study of up to 12 weeks in duration in subjects completing Cohorts 2, 3 and 4 of the parent study described in Example 2. The Parent study was a 12-week, multicenter study designed to assess the safety and tolerability of a single dose of CLS-AX administered suprachoroidally in subjects with neovascular age-related macular degeneration (wet AMD) who showed stable visual acuity following 3 or more injections with an intravitreal (IVT) anti-VEGF therapy in the preceding 5 months.

The primary objective of this study was to assess the safety and tolerability of a single dose of CLS-AX in participants with neovascular age-related macular degeneration up to 24 weeks following administration of CLS-AX at Visit 2 (Baseline, Day 1) in the Parent study.

The secondary objective was to evaluate the effect of a single dose of CLS-AX on visual function and ocular anatomy and the need for additional treatment for symptoms of wet AMD up to 24 weeks following administration of CLS-AX at Visit 2 (Baseline, Day 1) in the Parent study. The study included the following 3 dose cohorts:
Cohort 2 (low-mid dose arm): 0.10 mg suprachoroidal injection of CLS-AX
Cohort 3 (high-mid dose arm): 0.50 mg suprachoroidal injection of CLS-AX
Cohort 4 (high dose arm): 1.0 mg suprachoroidal injection of CLS-AX

Subjects who were administered by suprachoroidal injection in cohorts of the parent study (described in Example 5), were followed for an additional 12 weeks following exit from the Parent study. No intervention was administered in this extension study.

### Primary Outcome Measures:

1. The number of subjects experiencing treatment-emergent adverse events (TEAEs) (Time Frame: 24 weeks): The number of subjects experiencing treatment-emergent adverse events (TEAEs). Treatment-emergent adverse events were defined as an event that emerges following treatment with CLS-AX (administered at Visit 2 (Baseline, Day 1) of the Parent study, having been absent pre-treatment or worsens relative to the pre-treatment state.
2. The number of subjects experiencing serious adverse events (SAEs) (Time Frame: 24 weeks): The number of subjects experiencing serious adverse events (SAEs). Serious adverse events are defined as a serious event that emerges following treatment with CLS-AX (administered at Visit 2 (Baseline, Day 1) of the Parent study, having been absent pre-treatment or worsens relative to the pre-treatment state.

### Secondary Outcome Measures:

1. Counts and percentages and/or descriptive statistics of changes from Baseline in safety parameters including: intraocular pressure, slit-lamp biomicroscopy outcomes, ophthalmoscopy outcomes, and imaging parameters in the study eye; and vital signs at Visits 3, 4, 5, 6, 7 and 8 (Weeks 4, 8, 12, 16, 20 and 24).
2. Number and percentage of participants meeting the additional therapy criteria in the study eye at Visits 3, 4, 5, 6, 7 and 8 (Week 4, 8, 12, 16, 20 and 24).
3. Number and percentage of participants receiving additional therapy in the study eye at Visits 3, 4, 5, 6, 7 and 8 (Weeks 4, 8, 12, 16, 20 and 24).
4. Mean change from Baseline in best corrected visual acuity letter score in the study eye at Visits 3, 4, 5, 6, 7 and 8 (Weeks 4, 8, 12, 16, 20 and 24).
5. Mean change from Baseline in central subfield retinal thickness (CST) in the study eye at Visits 3, 4, 5, 6, 7 and 8 (Weeks 4, 8, 12, 16, 20 and 24).
   Inclusion Criteria: Enrolled in and completed the Parent study (Example 2) as part of Cohort 2, Cohort 3, or Cohort 4.
   Exclusion Criteria: Received prohibited medication in the Parent study; enrolled in the parent study as part of Cohort 1; or females of childbearing potential who are pregnant and or lactating.
   Additional Therapy Criteria: At Visit 6 (Week 16), Visit 7 (Week 20) and Visit 8 (Week 24) of this extension study, participants were evaluated for the need for additional therapy for wet AMD in the study eye based on the following criteria:
Loss of 10 or more letters in BCVA compared to the best prior Parent- and extension study assessed BCVA in the study eye that was attributed to intra- or sub-retinal fluid observed by the Investigator, OR
Increase in central subfield retinal thickness greater than 75 microns from the Parent study's Visit 2 (Baseline) in the study eye, OR
Presence of vision-threatening hemorrhage due to AMD in the study eye.

If the pre-defined criteria were met at Visits 6 and/or 7 (Weeks 16 and/or 20), then additional therapy consisting of an IVT injection of aflibercept 2 mg (0.05 mL), ranibizumab 0.5 mg (0.05 mL), bevacizumab 1.25 mg (0.05 mL), or brolucizumab 6 mg (0.05 mL), as determined by the Investigator, could be administered; even if additional therapy was given, the participants was to remain in the study and was to be followed until Visit 8 (Study Exit).

Biological Effect in the 6-Month Extension Study in Cohorts 3 and 4 (n=12)

There were no reports of death, SAEs, TEAEs considered related to CLS-AX or the injection procedure. Additionally, no safety trends were observed related to ocular inflammation, vasculitis, IOP, visual function, ocular anatomy or dispersion of drug into the vitreous.

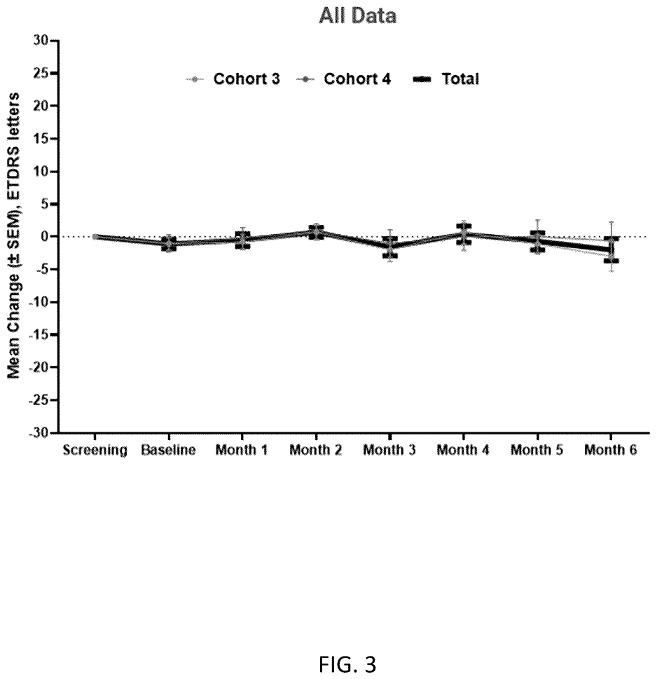

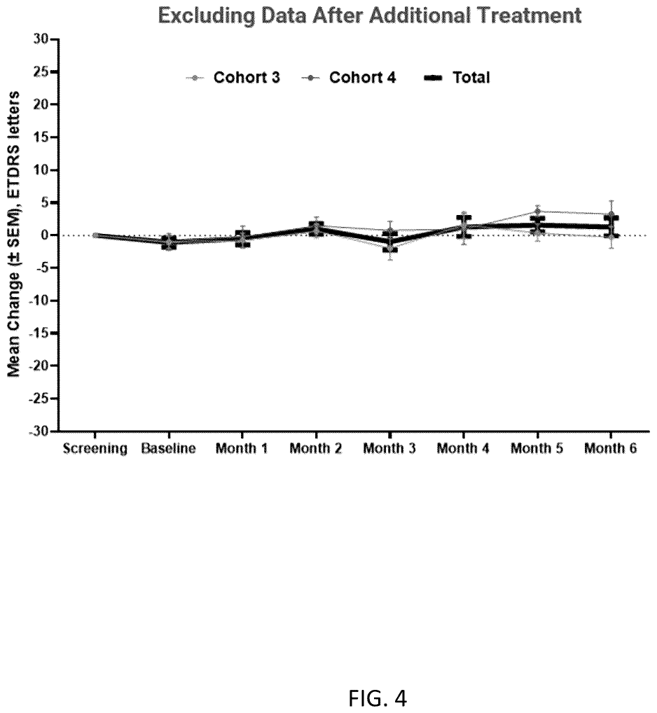

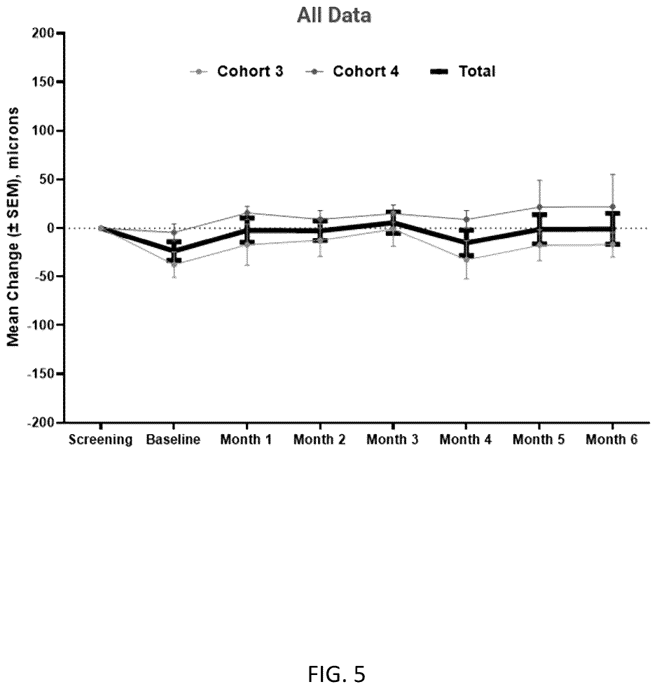

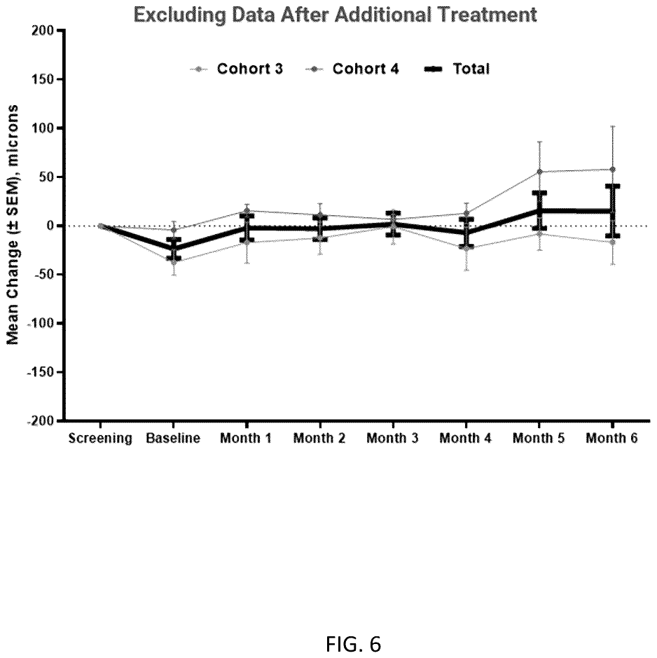

CLS-AX showed signs of biologic effect with stable mean best corrected visual acuity (BCVA) and stable mean central subfield thickness (CST) to the 6-month timepoint with mean changes ranging from approximately −2 to −2.5 letters and approximately 13 to 37 microns, respectively (FIGS. 3-6). On Optical Coherence Tomography (OCT) images, anatomical signs of TKI biologic effect were observed in anti-VEGF treatment experienced sub-responders.

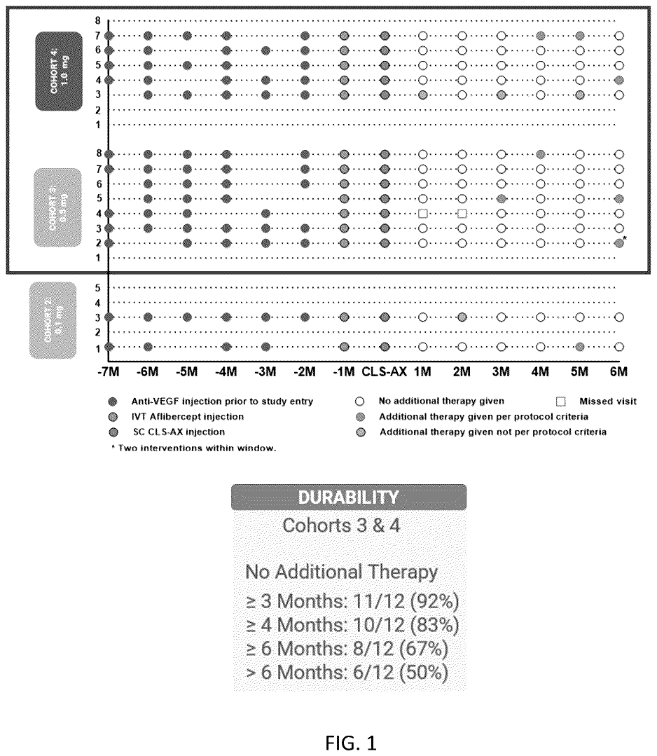

Durability in cohorts 3 and 4 at higher doses: 77%-85% reduction in treatment burden was observed compared to the average monthly injections in the six months before CLS-AX administration. As shown in FIG. 1, The participants who did not require additional therapies were:
≥3 Months: 11/12 (92%)
≥4 Months: 10/12 (83%)
≥6 Months: 8/12 (67%)
>6 Months: 6/12 (50%)

The CLS-AX administration demonstrated reduction of treatment burden (77-88% reduction) across all tested cohorts, as shown in Tables 2 and 3.

TABLE 2
Observed reduction in treatment burden (All Therapies)

| | | Avg Monthly | Avg Monthly | |
|---|---|---|---|---|
| | | Injections Before | Injections | |
| | | CLS-AX | After CLS-AX | % |
| Cohort | Participants | Administration | Administration | Reduction |
| 4 | 5 | 0.87 | 0.20 | 77.0 |
| 3 | 7 | 0.81 | 0.12 | 85.2 |
| 2 | 2 | 0.83 | 0.17 | 79.5 |

TABLE 3
Observed reduction in treatment burden
(Therapies per protocol criteria)

| | | Avg Monthly | Avg Monthly | |
|---|---|---|---|---|
| | | Injections Before | Injections | |
| | | CLS-AX | After CLS-AX | % |
| Cohort | Participants | Administration | Administration | Reduction |
| 4 | 4 | 0.83 | 0.13 | 84.0 |
| 3 | 7 | 0.81 | 0.12 | 85.2 |
| 2 | 1 | 0.67 | 0.17 | 74.6 |

### Example 4. A Clinical Study of Suprachoroidally Administered CLS-AX in Participants with Neovascular Age-Related Macular Degeneration (Wet AMD)

This study was a multi-center, randomized, double-masked clinical trial to assess the safety and efficacy of CLS-AX in the treatment of neovascular age-related macular degeneration (Wet AMD). Participants were diagnosed with neovascular AMD within 36 months of screening with confirmed persistent active disease with BCVA reading of 20 to 80 letters. Participants were randomized 1:1 to receive either CLS-AX administered by suprachoroidal injection via SCS microinjector or intravitreal aflibercept. Participants in both arms (CLRS-AX and aflibercept control group) received three loading doses of aflibercept (2 mg each). At baseline visit, the CLRS-AX arm received a single dose of 1.0 mg CLS-AX. CLS-AX arm was designed to receive dose at least every 24 weeks, unless addition dose was needed in between, while aflibercept arm received dose every 8 weeks.

The mean age of participants was 76.9 (51-90) years old and 80.3 (54-96) years old for CLS-AX arm and aflibercept arm, respectively. Participants One of the main objectives of the trial was to demonstrate comparable mean change in BCVA from baseline between treatment arms with improved durability and reduced treatment burden for the CLS-AX arm, measured at 6 and 12 months.

Primary objective: The primary objective of the study was to assess the efficacy of CLS-AX on best corrected visual acuity (BCVA) in participants diagnosed with wet AMD and to characterize the safety and tolerability of CLS-AX.
Secondary objectives: The secondary objectives of this study were to evaluate the efficacy of CLS-AX on additional BCVA outcomes, to evaluate the frequency of CLS-AX administration, and to determine the anatomic effects of CLS-AX.
Exploratory objective of this study was to evaluate the safety and performance of the SCS microinjector via AE reporting and investigator questionnaire.

### Study Design

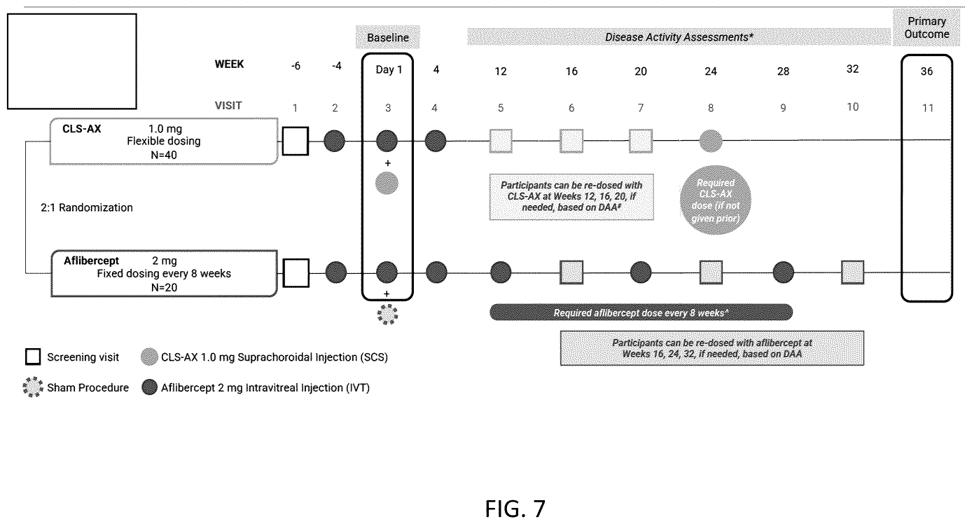

The study was designed to assess the safety and efficacy of CLS-AX 1.0 mg 36 weeks duration administered via SCS injection in participants diagnosed with choroidal neovascularization (CNV) secondary to age-related macular degeneration (AMD) who have been previously treated for wet AMD in the study eye. Initial eligibility was determined based on evaluations performed at Visit 1 (Screening, up to Day −42). All eligible participants will receive standard loading doses of IVT aflibercept 2 mg in the study eye at Visit 2 (Day −28), Visit 3 (Baseline, Day 1), and Visit 4 (Week 4). At Visit 3 (Baseline, Day 1), qualifying participants were randomly assigned in a 2:1 ratio to 1 of 2 treatment arms, CLS-AX 1.0 mg or aflibercept 2 mg. From Visit 3 (Baseline, Day 1), participants were followed for up to 36 weeks. The expected total duration of participation in the study was up to 36 weeks. Detailed study design is illustrated in FIG. 7.

Treatment Group (CLS-AX): Participants randomized to CLS-AX received an IVT injection of 2 mg of aflibercept in conjunction with a dose of 1.0 mg of CLS-AX injected suprachoroidally via the SCS microinjector in the study eye. Beginning at Visit 5 (Week 12) and at every scheduled visit through Visit 10 (Week 32), a disease activity assessment was performed on the study eye to determine whether a CLS-AX or sham dose should be administered, but the dose may be given only if the prior dose of CLS-AX occurred at least 12 weeks from the current dose. A dose of CLS-AX was administered, irrespective of disease activity, if 24 weeks had passed since the previous dose of CLS-AX was administered.

Treatment Group (Aflibercept Control) At Visit 3 (Baseline, Day 1), those participants randomized to aflibercept received an IVT injection of aflibercept 2 mg in conjunction with a sham procedure in the study eye. Beginning at Visit 5 (Week 12) and at every scheduled visit through Visit 10 (Week 32), a disease activity assessment was performed on the study eye to determine whether aflibercept or sham should be administered. Participants were assigned to a Q8 week fixed dosing schedule. A sham procedure was performed at those visits in which aflibercept was not otherwise scheduled or indicated by the disease activity assessment.

Inclusion Criteria: Participants were eligible for participation in this study if s/he met ALL the following criteria at Visit 1 (Screening):
1. Diagnosis of neovascular age-related macular degeneration in the study eye within 36 months of Visit 1 (Screening).
2. Subfoveal choroidal neovascularization (CNV) secondary to wet AMD of any lesion type in the study eye at Visit 1 (Screening) with photographs and/or fluorescein angiography and/or spectral-domain optical coherence tomography (SD-OCT), that shows: Total lesion area (including CNV, hemorrhage, fibrosis, atrophy) ≤30 mm²; CNV component area of ≥50% of the total lesion area; or CNV must not be associated with subfoveal hemorrhage, subfoveal fibrosis or subfoveal atrophy.
3. Previous treatment in the study eye with between 2 and 4 anti-VEGF IVT injections for wet AMD (faricimab, ranibizumab, bevacizumab, brolucizumab, or aflibercept) per standard of care within 6 months of Visit 1 (Screening).
4. History of response to prior IVT anti-VEGF treatment since diagnosis, as evidenced at Visit 1 (Screening) by the following: overall decrease in wet AMD disease activity detected on SDOCT, assessed by the Investigator, AND stable or improved visual acuity.
5. Early Treatment Diabetic Retinopathy Study (ETDRS) BCVA of 20 to 80 total letters, inclusive (approximate Snellen equivalent, ≥20/400 to 20/25) in the study eye.
6. At least 50 years of age.

### Exclusion Criteria:

Ophthalmic exclusion criteria: Participants were ineligible for participation in this study if s/he met any of the following criteria at Visit 1 (Screening) in the study eye:
1. ETDRS BCVA <20 total letters (approximate Snellen equivalent, less than 20/400).
2. Central subfield retinal thickness >400 microns, or retinal pigment epithelium detachment thickness >400 microns on SD-OCT.
3. FFA/color fundus photography (CFP): a. Subretinal hemorrhage of >50% of the total lesion area and/or that involves the fovea; b. Fibrosis or atrophy of >50% of the total lesion area and/or that involves the fovea.
4. CNV due to causes other than AMD, such as ocular histoplasmosis, trauma, pathological myopia, angioid streaks, choroidal rupture, or uveitis.
5. Any history of macular pathology unrelated to AMD affecting vision or contributing to the presence of intraretinal or subretinal fluid.
6. Presence of central serous chorioretinopathy.
7. Retinal pigment epithelial tear involving the macula.
8. Any concurrent intraocular condition (e.g., amblyopia, aphakia, retinal detachment, cataract, diabetic retinopathy or maculopathy, or epiretinal membrane with traction) that, in the opinion of the Investigator, could either reduce the potential for visual improvement or require medical or surgical intervention during the study.
9. Current vitreous hemorrhage.
10. Intraocular pressure (IOP) ≥25 mmHg or cup-to-disc ratio >0.8.
11. Spherical equivalent of refractive error demonstrating more than 6 diopters of myopia.
12. Any prior or concomitant treatment for CNV or vitreomacular-interface abnormalities, including, but not restricted to, IVT treatment (e.g., steroids, tissue plasminogen activator, ocriplasmin, C3F8 air), periocular pharmacological intervention, argon laser photocoagulation, verteporfin photodynamic therapy, diode laser, transpupillary thermotherapy, or ocular surgical intervention. Note: anti-VEGF IVT injections for wet AMD (faricimab, ranibizumab, bevacizumab, brolucizumab, or aflibercept) per standard of care are acceptable.
13. Any cataract surgery or treatment within 3 months prior to Visit 1.
14. Any other intraocular surgery (e.g., pars plana vitrectomy, minimally invasive glaucoma surgery, glaucoma laser procedures or surgery, corneal transplant, or radiotherapy) within 3 months prior to Visit 1.
15. Prior periocular pharmacological or IVT treatment (including anti-VEGF medication) for other retinal diseases.
16. Concomitant therapy with any drug that may be toxic to the lens, retina, or optic nerve, such as hydroxychloroquine, chloroquine, pentosan polysulfate, fingolimod, and tamoxifen.
17. Non-functioning non-study eye, defined as either: BCVA of hand motion or worse, no physical presence of non-study eye.
18. History of idiopathic or autoimmune-associated uveitis in either eye.
19. Active ocular inflammation or suspected or active ocular or periocular infection in either eye.
   Randomized Criteria: Participants were eligible for randomization if s/he met the following criteria at Visit 3 (Baseline, Day 1):
1. Less than or equal to 7 letter change in BCVA between Visit 1 (Screening) and Visit 3 (Baseline, Day 1) in the study eye.
2. No active hemorrhage in the study eye due to wet AMD.
3. The participant does not have a new health condition or a worsening existing condition that requires care or medication prohibited by the protocol.

Endpoints: The primary efficacy endpoint was the mean change in BCVA at Visit 11 (Week 36) from baseline (Day 1). Other primary endpoints included proportion of participants gaining ≥15, ≥10, ≥5, or ≥0 letters in BCVA over time and proportion of participants avoiding loss of ≥15, ≥10, ≥5, or >0 letters in BCVA over time. Key secondary endpoints included: 1) mean change in central subfield retinal thickness (CST) over time; 2) proportion of participants with absence of intraretinal fluid over time; 3) proportion of participants with absence of subretinal fluid over time; 4) proportion of participants with absence of intraretinal and subretinal fluid over time; 5) proportion of participants with absence of intraretinal cysts over time; 6) proportion of participants with absence of pigment epithelial detachment over time; 7) mean change from Visit 1 (Screening) in the total area of CNV at Visit 11 (Week 36); 8) mean change from Visit 1 (Screening) in the total lesion area at Visit 11 (Week 36); 9) mean change from Visit 1 (Screening) in the total area of CNV leakage at Visit 11 (Week 36); and 10) number of study drug injections received through Visit 10 (Week 32); 11) number of supplemental therapies administered through Visit 10 (Week 32). Endpoints for determining the safety and tolerability of CLS-AX included assessing the number of participants with TEAEs and SAEs considered related to the injection procedure.

### Safety Assessment

There were no reports of death, SAEs, TEAEs considered related to CLS-AX or the injection procedure through 36-week study period. Additionally, no safety trends were observed related to ocular inflammation, vasculitis, IOP, visual function, ocular anatomy or dispersion of drug into the vitreous. No drug or procedure related ocular or systemic SAE or TEAE was observed. No endophthalmitis or retinal vasculitis was observed.

### Efficacy Assessment

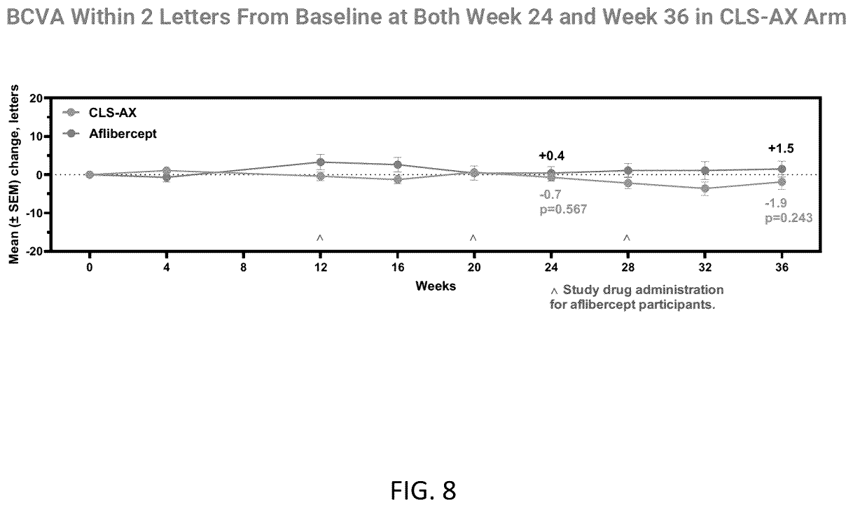

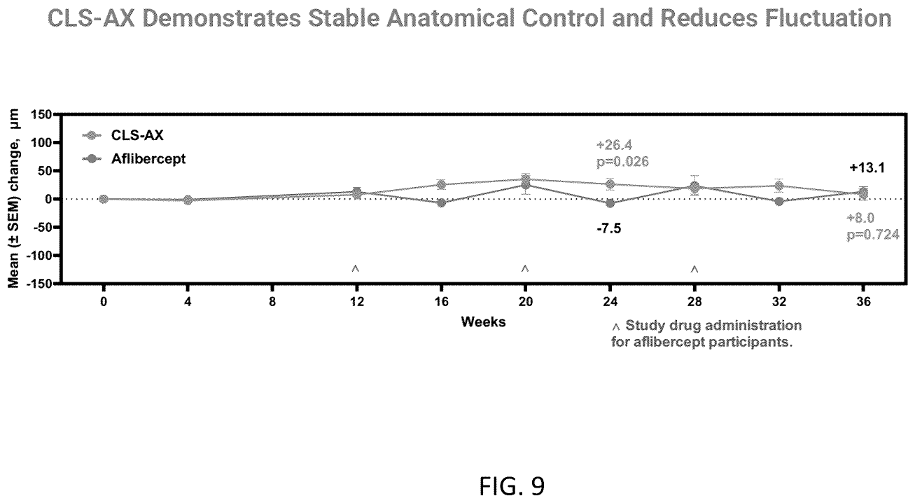

CLS-AX showed positive efficacy data in treating Wet AMD. Stable BCVA and CST values were observed throughout the trial as determined by measuring the mean change in BCVA from baseline to Week 36 between two arms (CLS-AX arm and aflibercept arm). There was no statistically significant difference in mean changes in BCVA and CST from baseline to Week 36 between the two arms (FIGS. 8 and 9).

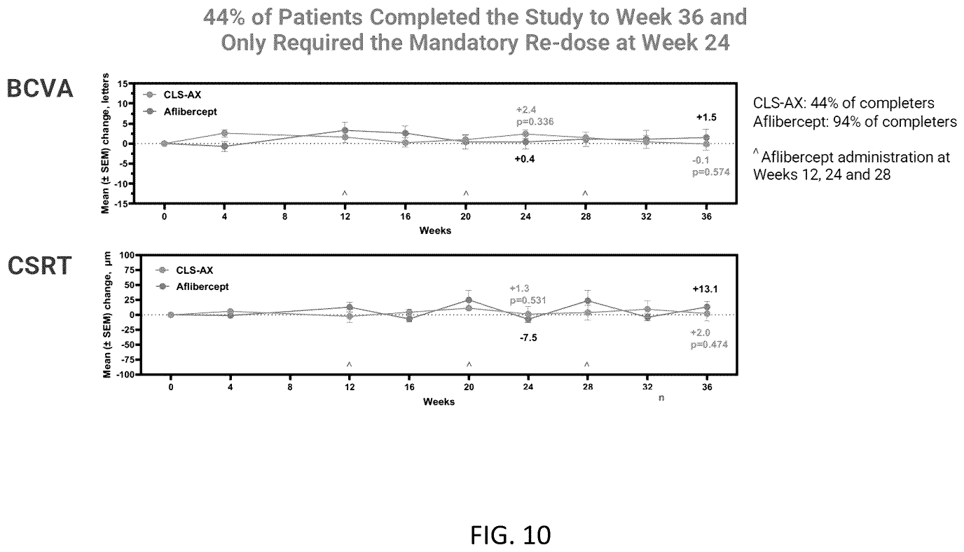

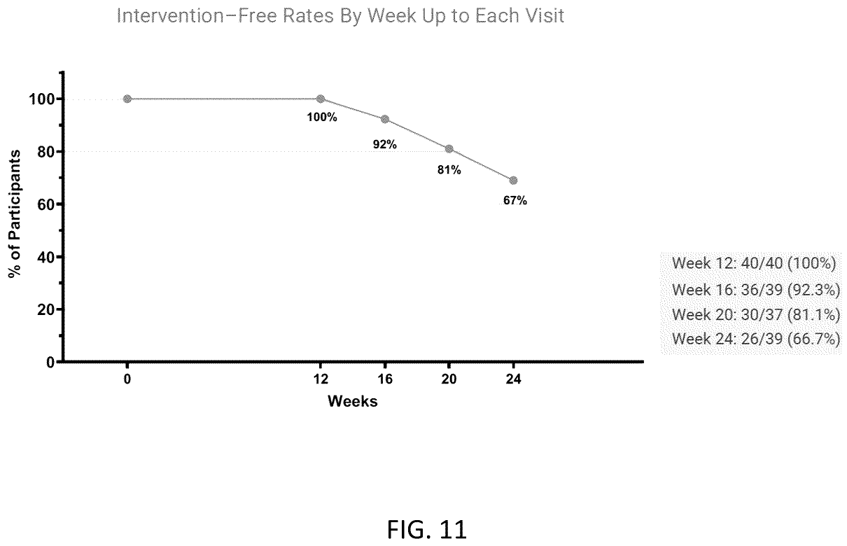

Importantly, as shown in FIG. 10, 44% of participants in the CLS-AX arm completed the study at Week 36 without requiring supplemental therapy. Furthermore, two-thirds of participants dosed with CLS-AX exhibited six months of intervention-free durability (FIG. 11). The % of participants who did not require additional therapies are summarized below:
92% of patient in CLS-AX did not require treatment for 16 weeks (4 months)
81% of patient in CLS-AX did not require treatment for 20 weeks (5 months)
67% of patient in CLS-AX did not require treatment for 24 weeks (6 months) before mandatory re-dosing at Week 24.
Overall injection frequency was reduced by about 84% (reduction in treatment burden) compared to the average monthly injections in the 24 weeks prior to Screening.

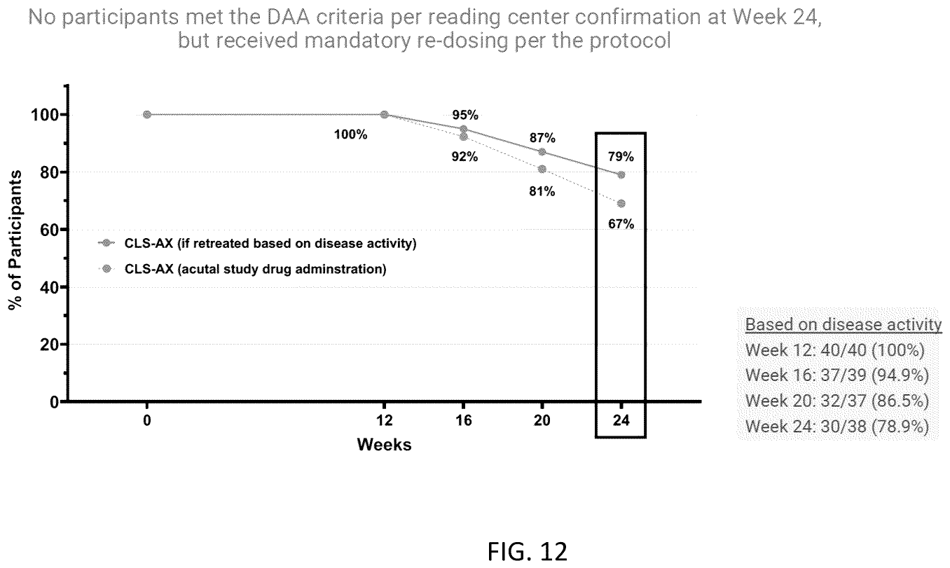

At Week 12 through Week 32, participates in both arms underwent Disease Activity Assessment (DAA) to determine if there was a need for supplemental treatment. Supplemental treatment criteria included decrease in BCVA (reduction of >10 letters from baseline), increase in CST (increase of >100 microns on SD-OCT from baseline), BCVA reduction of <5 letters from baseline and increase in CST of >75 microns from baseline, or new or worsening vision-threatening hemorrhage due to wet AMD. Although no DAA criteria for supplemental treatment at Week 24 was observed, all participants in CLS-AX arm received a mandatory dose of CLS-AX per the study protocol. More participants intervention free at every time point would have reached if DAA criteria were strictly applied, as shown in FIG. 12 and summarized below:
100% of patient in CLS-AX did not require treatment for 12 weeks (3 months) if retreated based on DAA criteria
95% of patient in CLS-AX did not require treatment for 16 weeks (4 months) if retreated based on DAA criteria
87% of patient in CLS-AX did not require treatment for 20 weeks (5 months) if retreated based on DAA criteria
79% of patient in CLS-AX did not require treatment for 24 weeks (6 months) if retreated based on DAA criteria

The study results indicate that CLS-AX consistently reduced the frequency of injections as illustrated in Table 4.

TABLE 4
Comparison of Wet AMD Treatments
Pre- and Post-Randominzation

| | Prior to Screening | After Baseline |
|---|---|---|
| Average number of treatment | 2.95 injections | 0.475 injections |
| (injections), 24 Weeks | | |

Reduced injection frequency by 83.9%

### Example 5. Clinical Study of Suprachoroidally Administered CLS-AX in Participants with Neovascular Age-Related Macular Degeneration (Wet AMD)

This study is a clinical trial to assess the efficacy of CLS-AX in the treatment of neovascular age-related macular degeneration (Wet AMD). Participants who are diagnosed with neovascular AMD but have not previously treated with any of the study drugs (treatment-naïve) will be randomized into CLRS-AX and aflibercept control groups. At baseline visit, the CLRS-AX arm will receive a single dose of 1.0 mg CLS-AX injected suprachoroidally via the SCS microinjector in the study eye while aflibercept arm will receive 2.0 mg of aflibercept (or faricimab) via intravitreal injection. Beginning at Week 12 and at every scheduled visit through Week 32, a disease activity assessment will be performed on the study eye to determine whether additional maintenance dose should be administered. The efficacy and durability of CLS-AX maintenance dosing regimen between 3 months and 6 months for treating Wet AMD will be assessed.

1. A method of treating neovascular age-related macular degeneration (wet AMD), comprising non-surgically administering a formulation comprising an effective amount of axitinib to the suprachoroidal space (SCS) of the eye of a human patient in need thereof, wherein the effective amount of axitinib in the formulation is about 1.0 mg.

2. The method of claim 1, wherein the formulation comprising the effective amount of axitinib is administered in a first dose.

3. The method of claim 2, wherein the method further comprises administering one or more maintenance doses.

4. The method of claim 3, wherein at least one of the one or more maintenance doses is administered about 2 months to about 8 months following the first dose.

5. The method of claim 4, wherein at least one of the one or more maintenance doses is administered about 3 months to about 6 months following the first dose.

6. The method of claim 4, wherein at least one of the one or more maintenance doses is administered at about 3 months, at about 4 months, at about 5 months, or at about 6 months, following the first dose.

7. The method of claim 3, wherein at least one of the one or more maintenance doses is administered about every 4-36 weeks following the first dose.

8. The method of claim 7, wherein at least one of the one or more maintenance doses is administered about every 6-32 weeks following the first dose.

9. The method of claim 7, wherein at least one of the one or more maintenance doses is administered about every 8-28 weeks following the first dose.

10. The method of claim 7, wherein at least one of the one or more maintenance doses is administered about every 12-24 weeks following the first dose.

11. The method of claim 7, wherein at least one of the one or more maintenance doses is administered about every 24 weeks.

12. The method of claim 3, wherein the method reduces injection frequency for the human patient within about 24 weeks to about 36 weeks following the first dose compared to a human patient who is not administered the formulation comprising the effective amount of axitinib to the SCS.

13. The method of claim 12, wherein the method reduces injection frequency for the human patient within about 24 weeks to about 36 weeks following the first dose compared to the injection frequency of the human patient prior to administration of the formulation.

14. The method of claim 12, wherein the injection frequency is reduced to about 30-90% fewer doses.

15. The method of claim 12, wherein the injection frequency is reduced to about 1-4 fewer injections.

16. The method of claim 12, wherein the human patient does not require a maintenance dose within about 3 months, 4 months, or 6 months following the first dose, determined by Disease Activity Assessment (DAA).

17. The method of claim 12, wherein the human patient requires only one maintenance dose within about 3 months, 4 months, or 6 months following the first dose, determined by Disease Activity Assessment (DAA).

18. The method of claim 1, wherein the human patient does not require an additional VEGF therapy following about 3 months, 4 months, or 6 months following the last administration of the formulation, determined by Disease Activity Assessment (DAA).

19-30. (canceled)

31. The method of claim 1, wherein the human patient loses no more than 10 letters or no more than 5 letters as measured by best-corrected visual acuity (BCVA) measurement, compared to the patient's BCVA measurement prior to administration of the formulation.

32-35. (canceled)

36. The method of claim 1, wherein the patient experiences minimal loss in vision for 2, 3, 4, 5, 6, 7, 8, or 9 months after administration of the formulation comprising axitinib, wherein the minimal loss of vision is losing no more than 2 letters or no more than 5 letters as measured by BCVA.

37-46. (canceled)
